(19) United States
(12) Patent Application Publication
(10) Pub. No.: US 2007/0275410 A1
(43) Pub. Date: Nov. 29, 2007

(54) Methods and Compositions Relating to Modulation of Gpcr Signaling

(75) Inventors: Fu-Yue Zeng, San Diego, CA (US); Dominic Behan, San Diego, CA (US)

Correspondence Address: ARENA PHARMACEUTICALS, INC., 6166 NANCY RIDGE DRIVE, SAN DIEGO, CA, 92121, US

(73) Assignee: Arena Pharmaceuticals, Inc., San Diego, CA (US)

(21) Appl. No.: 10/568,301

(22) PCT Filed: Aug. 9, 2004
(86) PCT No.: PCT/US04/25854

§ 371 Date: Jan. 26, 2007

Related U.S. Application Data
(60) Provisional application No. 60/496,255, filed on Aug. 18, 2003.

(51) Int. Cl. G01N 33/74; C07H 21/04; C07K 16/18; C12N 5/06

(52) U.S. Cl. 435/7.1; 435/325; 530/387.1; 536/23.1; 536/23.5

(57) ABSTRACT

The subject invention provides methods for detecting a GPCR-binding partner complex. In general, the methods involve co-producing two polypeptides, one of which being a GPCR, isolating one of the polypeptides using a substrate with affinity for that polypeptide, and directly detecting the presence of the other polypeptide on the substrate. In some embodiments, the affinity substrate is addressable. The two polypeptides may be the same or different GPCRs, or a GPCR and a non-GPCR polypeptide. The subject invention further provides methods for identifying whether a polypeptide is a binding partner for a GPCR. In addition, the subject invention provides methods of screening for agents that modulate the binding of a GPCR to a binding partner for that GPCR. The subject methods and compositions find use in a variety of research and therapeutic applications, particularly in methods to identify agents for treating GPCR-binding partner complex-related disorders.

This application claims the benefit of priority from the following provisional application, filed via U.S. Express mail with the United States Patent and Trademark Office on the indicated date: U.S. Provisional No. 60/496,255, filed Aug. 18, 2003. The disclosure of the foregoing application is herein incorporated by reference in its entirety.

### DESCRIPTION

### Field of the Invention

The present invention generally relates to methods and compositions for use in identifying agents that modulate G protein-coupled receptor (GPCR) activity. Specifically, the invention relates to methods for identifying a binding partner for a GPCR, and methods for modulating the interaction between the GPCR and its binding partner.

### BACKGROUND OF THE INVENTION

GPCR signaling plays a vital role in a number of physiological contexts including, but not limited to, metabolism, inflammation, neuronal function, and cardiovascular function. For instance, by way of illustration and not limitation, GPCRs include receptors for biogenic amines, e.g., dopamine, epinephrine, histamine, glutamate, acetylcholine, and serotonin; for purines such as ADP and ATP; for the vitamin niacin; for lipid mediators of inflammation such as prostaglandins, lipoxins, platelet activating factor, and leukotrienes; for peptide hormones such as calcitonin, follicle stimulating hormone, gonadotropin releasing hormone, ghrelin, motilin, neurokinin, and oxytocin; for non-hormone peptides such as beta-endorphin, dynorphin A, Leu-enkephalin, and Met-enkephalin; for the non-peptide hormone melatonin; for polypeptides such as C5a anaphylatoxin and chemokines; for proteases such as thrombin, trypsin, and factor Xa; and for sensory signal mediators, e.g., retinal photopigments and olfactory stimulatory molecules.

GPCRs represent an important area for the development of pharmaceutical products: approximately 60% of all prescription pharmaceuticals have been developed from approximately 20 of the 100 known GPCRs. For example, in 1999, of the top 100 brand name prescription drugs, the following drugs interact with GPCRs (the primary disease and/or disorder treated related to the drug is indicated in parentheses): Claritin® (allergies), Prozac® (depression), Vasotec® (hypertension), Paxil® (depression), Zoloft® (depression), Zyprexa® (psychotic disorder), Cozaar® (hypertension), Imitrex® (migraine), Zantac® (reflux), Propulsid® (reflux disease), Risperdal® (schizophrenia), Serevent® (asthma), Pepcid® (reflux), Gaster® (ulcers), Atrovent® (bronchospasm), Effexor® (depression), Depakote® (epilepsy), Cardura® (prostatic hypertrophy), Allegra® (allergies), Lupron® (prostate cancer), Zoladex® (prostate cancer), Diprivan® (anesthesia), BuSpar® (anxiety), Ventolin® (bronchospasm), Hytrin® (hypertension), Wellbutrin® (depression), Zyrtec®, (rhinitis), Plavix® (MI/stroke), Toprol-XL® (hypertension), Tenormin® (angina), Xalatan® (glaucoma), Singulair® (asthma), Diovan® (hypertension) and Harnal® (prostatic hyperplasia) (Med Ad News 1999 Data).

GPCRs share a common structural motif, having seven sequences of between 22 to 24 hydrophobic amino acids that form seven alpha helices, each of which spans the membrane (each span is identified by number, i.e., transmembrane-1 (TM1), transmembrane-2 (TM2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC1, EC2 and EC3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC1, IC2 and IC3), respectively). The “carboxy” (“C”) terminus of the receptor lies in the intracellular space within the cell, and the “amino” (“N”) terminus of the receptor lies in the extracellular space outside of the cell.

Generally, when a ligand binds with the receptor (often referred to as “activation” of the receptor), there is a change in the conformation of the receptor that facilitates coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz, Go, G11, G12, G13, G15 and G16 are G proteins that have been identified. Coupling with Gq, G11, G15 or G16 leads to an increase in intracellular IP3 concentration and an increase in intracellular Ca²⁺ concentration. Coupling to Gs leads to an increase in intracellular cAMP concentration. Coupling to Gi, Go, or Gz leads to a decrease in intracellular cAMP concentration. Ligand-activated GPCR coupling with a G-protein initiates a signaling cascade process (referred to as “signal transduction”). Under normal conditions, signal transduction ultimately results in cellular activation or cellular inhibition.

As discussed above, GPCRs interact with G-proteins to initiate a signal. As well as interacting with G-proteins, GPCRs can homodimerize (e.g., Floyd et al, JBC July, 2003), heterodimerize (e.g., Xu et al, JBC 278: 10770-10777, 2003), and interact with other proteins in a cell (e.g., “RAMPS”, see Sexton et al, Cell. Sign. 13:73-83, 2001); (Agnati et al., Pharmacol Rev. 55:509-550, 2003; the disclosure of which is hereby incorporated by reference in its entirety). Binding of a GPCR to its binding partners has been proposed to modulate signaling by the GPCR.

It follows from the foregoing that compounds that specifically modulate the binding of a GPCR to a binding partner may modulate signaling by that GPCR, and, as such, are extremely desirable. Accordingly, there is a great need for methods and compositions for identifying GPCR binding partners, and identifying compounds that can modulate the interaction between a GPCR and a binding partner for the GPCR. It also follows that identifying GPCR binding partners allows for the characterization of the pharmacology of the GPCR in the presence and absence of a binding partner for the GPCR and for the design of novel screening assays directed to modulators of the GPCR-binding partner-complex functionality. In particular, there is a great need for widely applicable, effective, high-throughput and sensitive assays for screening candidate binding partners for binding to a GPCR, and to identify compounds that modulate the binding of a GPCR to a binding partner for that GPCR. This invention meets these, and other, needs.

Literature

Literature of, interest includes: Klco et al. J Biol Chem. 2003 Jun. 30 [Epub ahead of print] Floyd et al. J Biol Chem. 2003 Jun. 30 [Epub ahead of print]; Xu et al. J Biol Chem. 2003 278:10770-7; Rios et al. Pharmacol Ther. 2001 92:71-87; Sexton et al. Cell Signal. 2001 13:73-83; Joost et al. Genome Biol. 2002 Oct. 17; 3 (11):RESEARCH0063; Angers et al. Annu Rev Pharmacol Toxicol. 2002; 42:409-35; Rios et al. Pharmacol Ther. 2001 92:71-87; Hilairet et al. J Biol Chem. 2003; 278:23731-7; McVey et al. J Biol Chem. 2001 276:14092-9; and Agnati et al. Pharmacol Rev. 2003 55:509-550.

### SUMMARY OF THE INVENTION

The subject invention provides methods for detecting a GPCR complex. In general, the methods involve co-producing two polypeptides, one of which being a GPCR, isolating one of the polypeptides using a substrate with affinity for that polypeptide, and directly detecting the presence of the other polypeptide on the substrate. In some embodiments, the affinity substrate is addressable. The two polypeptides may be the same or different GPCRs, or a GPCR and a non-GPCR polypeptide. In addition, the subject invention provides methods for identifying whether a polypeptide is a binding partner of a GPCR. In addition, the subject invention provides methods of screening for agents that modulate the binding of a GPCR to a binding partner for that GPCR. The subject methods and compositions find use in a variety of research and therapeutic applications, particularly in methods to identify agents for treating GPCR-binding partner complex-related disorders.

One feature of the subject methods is that the assay may be performed using an addressable affinity substrate, e.g., a 96-well plate, facilitating the high-throughput identification of GPCR complexes, e.g., as relates to the identification of, agents that modulate complex formation. Also, in many embodiments, the subject methods involve pairwise testing of members of a plurality of GPCRs to systematically identify GPCR complexes.

The subject invention provides screening methods that can identify a GPCR binding partner and screening methods that can detect agents that modulate the binding of a GPCR to a binding partner for that GPCR at significantly less cost and with fewer steps than alternative assays. In most embodiments, the assay is non-radioactive, highly sensitive, and amenable to high-throughput format.

### BRIEF DESCRIPTION OF THE DRAWINGS

FIG. 1 is a schematic figure showing an exemplary embodiment of the invention.

FIG. 2 is a bar graph showing detection of β₂AR dimer as anti-FLAG antibody-bound β₂AR-Rlu. FLAG-β₂AR/β₂AR-Rlu homodimer was specifically detected by anti-FLAG antibody in a receptor dose-dependent manner.

FIG. 3 is a bar graph showing a comparison of anti-HA antibody and anti-FLAG antibody on detection of β₂AR homodimer. Anti-FLAG antibody showed better specificity and higher sensitivity than anti-HA antibody in detecting β₂AR homodimer.

FIG. 4 is a bar graph showing the effect of digitonin concentration on detection of β₂AR homodimer. Digitonin up to 0.5% had no effect on stability of FLAG-β₂AR/β₂AR-Rlu homodimer.

FIG. 5 is a compilation of two bar graphs, A and B, showing a comparison of digitonin and Triton X-100 on the solubilization and detection of β₂AR homodimer. Panel (A) shows the effect of digitonin and Triton X-100 detergent concentration on the solubilization of FLAG-β₂AR/β₂AR-Rlu homodimer. Both digitonin (>0.2%) and Triton X-100 (>0.1%) efficiently solubilize β₂AR homodimer from whole cells. Panel (B) shows the effect of method of lysate preparation on the detection of β₂AR homodimer. For lysate prepared from either whole cells or isolated membrane, the fraction of β₂AR-Rlu detected as binding partner to FLAG-β₂AR was in the range of 18-25%, with the theoretical maximum value being 50%.

FIG. 6 is a bar graph showing that the β₂AR homodimer detected by methods of the subject invention forms in cells and not as a result of non-specific protein interaction during experimental procedures. Dimeric FLAG-β₂AR/β₂AR-Rlu could only be detected when FLAG-β₂AR and β₂AR-Rlu were co-transfected. No significant dimeric FLAG-β₂AR/β₂AR-Rlu could be detected when lysate from FLAG-β₂AR-expressing cells and lysate from β₂AR-Rlu-expressing cells were mixed. These results indicate that β₂AR dimerization occurs in intact cells.

FIG. 7 is a bar graph showing heterodimerization β₂AR with other GPCRs. α_{2A}AR, 5HT_{2C}, M₃, H₃, NPYR₁, 5HT_{2B} and H₂ could significantly associate with β₂AR, whereas GHSR, GPR50, D₂ and ADORA₁ had weak or no association with β₂AR. Each of the GPCRs displayed a human GPCR, except for α_{2A}AR, which is GPCR from pig.

FIG. 8 is a bar graph showing the effect of receptor expression levels on β2AR homo- and hetero-dimerization. Receptor expression levels were proportional to transfected receptor plasmid amount as confirmed by determining receptor binding sites in a radio-ligand binding assay. Physiological expression levels of β2AR in tissues or cells are in the range of 10-200 fmol per mg protein, which corresponds to conditions between “⅕” and “ 1/10” dilution. These results suggest that β₂AR can form dimers under physiological expression levels.

FIG. 9 is a compilation of graphs, A and B, showing the effect of 5HT_{2C} coexpression on agonist-stimulated cAMP production by β₂AR. Panels (A) and (B) show that coexpression of 5HT_{2C} potentiates agonist-stimulated cAMP production by β₂AR without significantly changing receptor expression level of β₂AR.

FIG. 10 is a bar graph showing heterodimerization of 5HT_{2C} with other GPCRs. 5HT_{2A}, β₂AR, β₃AR, M₁ and NPYR₅ could significantly associate with 5HT_{2C}, whereas other tested receptors had weak or no association with 5HT_{2C}. Each of the GPCRs displayed is human.

FIG. 11 shows detection of Gαs interaction with β2AR-Rlu. Specific interaction of β2AR-Rlu with Gαs could be detected using anti-Gαs antibody for capture. Digitonin concentration had an effect on the total bound β2AR-Rlu (panel A), but had no significant effect on the signal-to-noise ratio (panel B).

### DEFINITIONS

Before the present invention is further described, it is to be understood that this invention is not limited to particular embodiments described, as such may of course vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

It must be noted that as used herein and in the appended claims, the singular forms “a”, “and”, and “the” include plural referents unless the context clearly dictates otherwise. Thus, for example, reference to “an agent” includes a plurality of such agents, and reference to “the GPCR” includes reference to one or more GPCRs and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

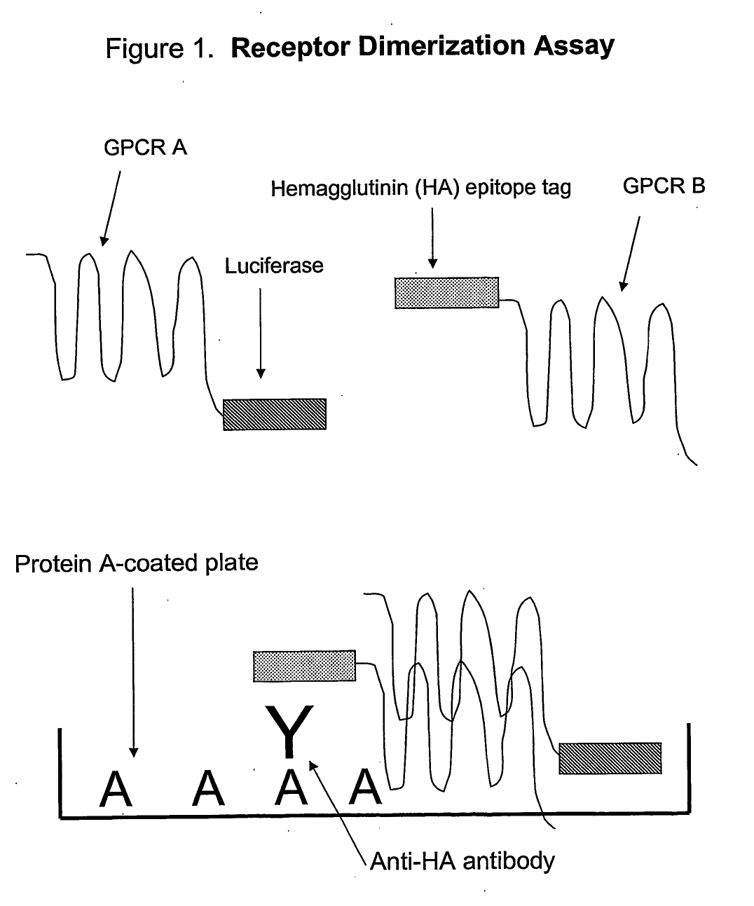

“G-protein coupled receptors”, or “GPCRs” are polypeptides that share a common structural motif, having seven regions of between 22 to 24 hydrophobic amino acids that form seven alpha helices, each of which spans a membrane [each span is identified by number, i.e., transmembrane-1 (TM1), transmembrane-2 (TM2), etc.]. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane [these are referred to as “extracellular” regions 1, 2 and 3 (EC1, EC2 and EC3), respectively]. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane [these are referred to as “intracellular” regions 1, 2 and 3 (IC1, IC2 and IC3), respectively]. The “carboxy” (“C”) terminus of the receptor lies in the intracellular space within the cell, and the “amino” (“N”) terminus of the receptor lies in the extracellular space outside of the cell. GPCR structure and classification is generally well known in the art, and further discussion of GPCRs may be found in Probst, DNA Cell Biol. 1992 11:1-20; Marchese et al Genomics 23: 609-618, 1994; and the following books: Jürgen Wess (Ed) Structure-Function Analysis of G Protein-Coupled Receptors published by Wiley-Liss (1st edition; Oct. 15, 1999); Kevin R. Lynch (Ed) Identification and Expression of G Protein-Coupled Receptors published by John Wiley & Sons (March 1998) and Tatsuya Haga (Ed), G Protein-Coupled Receptors, published by CRC Press (Sep. 24, 1999); and Steve Watson (Ed) G-Protein Linked Receptor Factsbook, published by Academic Press (1st edition; 1994). A schematic representation of a typical GPCR is shown in FIG. 1.

The term “endogenous” in reference to, for example and not limitation, a GPCR shall mean that which is naturally produced (for example and not limitation, by a mammal or by a human). As used herein, “endogenous GPCR” and “native GPCR” are interchangeably.

The term “orphan GPCR” shall mean an endogenous GPCR for which the native ligand specific for that GPCR has not been identified or is not known.

The term “ligand” means a molecule that specifically binds to a GPCR. A ligand may be, for example a polypeptide, a lipid, a small molecule, an antibody. A “native ligand” is a ligand that is an endogenous, natural ligand for a native GPCR. A ligand may be a GPCR “antagonist”, “agonist”, “partial agonist” or “inverse agonist”, or the like.

A “modulator” is a ligand that increases or decreases a GPCR intracellular response when it is in contact with, e.g., binds, to a GPCR that is expressed in a cell.

An “agonist” is a ligand which activates a GPCR intracellular response when it binds to a GPCR.

A “partial agonist” is a ligand what activates, to a lesser extent than an agonist, a GPCR intracellular response when it binds to a GPCR.

An “antagonist” is a ligand which competitively binds to a GPCR at the same site as an agonist but which does not activate the intracellular response produced by the active form of a GPCR. Antagonists usually inhibit intracellular responses by an agonist or partial agonist. Antagonists usually do not diminish the baseline intracellular response in the absence of an agonist or partial agonist.

An “inverse agonist” is a ligand which binds to a GPCR and inhibits the baseline intracellular response of the GPCR observed in the absence of an agonist or partial agonist. In most embodiments, a baseline intracellular response is inhibited in the presence of an inverse agonist by at least about 30%, by at least about 50%, or by at least 75%, as compared to a baseline response in the absence of an inverse agonist.

The term “constitutive GPCR activation” shall mean stabilization of a GPCR in the active state by means other than binding of the GPCR with its ligand or a chemical equivalent thereof. Constitutive GPCR activation typically is accomplished through site-specific mutation of the GPCR, comprising substitution of one or more amino acids or substitution of all or part of a domain (e.g., replacement of the endogenous IC3 loop with the IC3 loop from a different GPCR). In some embodiments, constitutive GPCR activation is accomplished through an algorithmic approach whereby the 16^{th} amino acid (located in the IC3 region of the GPCR) from a conserved proline (or an endogenous, conservative substitution therefore) residue (located in the TM6 region of the GPCR, near the TM6/IC3 interface) is mutated, preferably to an alanine, histidine, arginine, or lysine amino acid residue, most preferably to a lysine amino acid residue. (See, e.g., PCT Application Number PCT/US98/07496 published as WO 98/46995 on 22 Oct. 1998; U.S. Pat. No. 6,255,089; and U.S. Pat. No. 6,555,339; and the disclosure each of which is hereby incorporated by reference in its entirety.)

The term “constitutively activated GPCR” shall mean a receptor subjected to constitutive activation.

As used herein, the terms “GPCR-binding partner complex-related condition” and “GPCR-binding partner complex-related disorder” are used interchangeably to refer to any disorder, or symptoms of which, caused by or treatable by an alteration in the activity of a specific GPCR-binding partner complex. GPCR-binding partner complex-related conditions may be associated with aberrant activity of a GPCR in a complex, and may be caused by aberrant activity of a GPCR, such as in the cases where a GPCR is mutated or otherwise activated to cause an over-active, constitutively active, or under-active GPCR. Also within this definition are disorders treatable by altering the activity of a GPCR complex that has normal activity. For example, some disorders are not associated with the aberrant activity of a particular GPCR complex, but nevertheless are treatable by modulating that GPCR complex. Also encompassed by this term are cosmetic alterations, which are not life threatening but otherwise desirable to have. Exemplary GPCR complex-related conditions include allergies, hypertension, psychological disorders e.g. depression, anxiety and schizophrenia, migraine headaches, reflux, asthma and bronchospasm, prostatic hypertrophy, ulcers, epilepsy, angina, rhinitis, cancer e.g. prostate cancer, glaucoma and stroke. Further exemplary GPCR-related conditions at the On-line Mendelian Inheritance in Man database found at the world wide website of the NCBI.

The term “phenomenon associated with aberrant GPCR complex activity” as used herein refers to a structural, molecular, or functional characteristic associated with aberrant activity of a GPCR in a complex, particularly such a characteristic that is readily assessable in a human or animal model. Such characteristics include, but are not limited to, downstream molecular events caused by activation of a GPCR, and phenotypes or symptoms, for example, sneezing, nasal mucous production, acid reflux, mood, wheezing, pain, height, etc.

A “deletion” is defined as a change in either amino acid or nucleotide sequence in which one or more amino acid or nucleotide residues, respectively, are absent as compared to an amino acid sequence or nucleotide sequence of a parental GPCR polypeptide or nucleic acid. In the context of a GPCR or a fragment thereof, a deletion can involve deletion of about 2, about 5, about 10, up to about 20, up to about 30 or up to about 50 or more amino acids. A GPCR or a fragment thereof may contain more than one deletion.

An “insertion” or “addition” is that change in an amino acid or nucleotide sequence which has resulted in the addition of one or more amino acid or nucleotide residues, respectively, as compared to an amino acid sequence or nucleotide sequence of a parental GPCR. “Insertion” generally refers to addition to one or more amino acid residues within an amino acid sequence of a polypeptide, while “addition” can be an insertion or refer to amino acid residues added at an N- or C-terminus, or both termini. In the context of a GPCR or fragment thereof, an insertion or addition is usually of about 1, about 3, about 5, about 10, up to about 20, up to about 30 or up to about 50 or more amino acids. A GPCR or fragment thereof may contain more than one insertion.

A “substitution” results from the replacement of one or more amino acids or nucleotides by different amino acids or nucleotides, respectively as compared to an amino acid sequence or nucleotide sequence of a parental GPCR or a fragment thereof. It is understood that a GPCR or a fragment thereof may have conservative amino acid substitutions which have substantially no effect on GPCR activity. By conservative substitutions is intended combinations such as gly, ala; val, ile, leu; asp, glu; asn, gln; ser, thr; lys, arg; and phe, tyr.

The term “biologically active” GPCR refers to a GPCR having structural and biochemical functions of a naturally occurring GPCR.

As used herein, the terms “determining,” “measuring,” “assessing,” and “assaying” are used interchangeably and include both quantitative and qualitative determinations. Reference to an “amount” of a GPCR in these contexts is not intended to require quantitative assessment, and may be either qualitative or quantitative, unless specifically indicated otherwise.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term “fusion protein” or grammatical equivalents thereof is meant a protein composed of a plurality of polypeptide components, that while typically unjoined in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, preferably 75% free, and most preferably 90% free from other components with which it is naturally associated.

A “coding sequence” or a sequence that “encodes” a selected polypeptide, is a nucleic acid molecule which can be transcribed (in the case of DNA) and translated (in the case of mRNA) into a polypeptide, for example, in a host cell when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or prokaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. In the case of a promoter, a promoter that is operably linked to a coding sequence will effect the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to a host cell. Typically, “vector construct,” “expression vector,” and “gene transfer vector,” mean any nucleic acid construct capable of directing the expression of a gene of interest and which can transfer gene sequences to host cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into a host cell. Thus, the term includes cloning and expression vehicles, as well as viral vectors.

A first polynucleotide is “derived from” or “corresponds to” a second polynucleotide if it has the same or substantially the same nucleotide sequence as a region of the second polynucleotide, its cDNA, complements thereof, or if it displays sequence identity as described above.

A first polypeptide is “derived from” or “corresponds to” a second polypeptide if it is (i) encoded by a first polynucleotide derived from a second polynucleotide, or (ii) displays sequence identity to the second polypeptides as described above.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of GPCR-modulator that can provide for enhanced or desirable effects in the subject (e.g., reduction of pathogen load, beneficial increase in a physiological parameter of the subject, reduction of disease symptoms, etc.).

“Subject”, “individual,” “host” and “patient” are used interchangeably herein, to refer to an animal, human or non-human, susceptible to or having a GPCR-related disorder amenable to therapy according to the methods of the invention. Generally, the subject is a mammalian subject. Exemplary subjects include, but are not necessarily limited to, humans, non-human primates, mice, rats, cattle, sheep, goats, pigs, dogs, cats, and horses, with humans being of particular interest.

### DETAILED DESCRIPTION OF THE INVENTION

The subject invention provides methods for detecting a GPCR complex. In general, the methods involve co-producing two polypeptides, one of which being a GPCR, isolating one of the polypeptides using a substrate with affinity for that polypeptide, and detecting the presence of the other polypeptide on the substrate. In some embodiments, the affinity substrate is addressable, more particularly spatially addressable or spectrophotometrically addressable. The two polypeptides may be the same or different GPCRs, or a GPCR and a non-GPCR polypeptide. In addition, the subject invention provides methods of screening for agents that modulate the binding of a GPCR to a binding partner for that GPCR. The subject methods and compositions find use in a variety of research and therapeutic applications, particularly in methods to identify agents for treating GPCR-binding partner complex-related disorders.

In further describing the invention in greater detail than provided in the Summary and as informed by the Background and Definitions provided above, the compositions for use in the subject methods are described first, followed by a discussion of methods for identifying binding partners for a GPCR. This discussion is followed by a description of screening assays, a review of representative applications in which the subject methods find use, and subject kits provided for practicing the subject methods.

In general, in performing the subject methods, a GPCR is tested for binding to at least one member of a library of candidate polypeptides, which library may contain the same GPCR, a different GPCR, a GPCR accessory protein, or any other protein. In many embodiments, the GPCR and the candidate polypeptide are co-produced in a cell and the GPCR and the candidate polypeptide are tested for GPCR-binding partner complex formation. GPCR-binding partner complex formation is usually tested for by lysing the cell to make a cell extract, contacting the extract with an affinity substrate for either the GPCR or the candidate polypeptide, and detecting the presence of the other member of the complex. In many embodiments, the affinity substrate is an addressable affinity substrate. Once a GPCR-binding partner complex has been detected, the identity of the candidate polypeptide that forms a complex with the GPCR usually becomes known, and, as such, the methods provide a way of identifying binding partners for a GPCR that, in many embodiments, is a pre-selected GPCR.

Polypeptides of Interest

In general, the invention involves testing for binding between a GPCR polypeptide and at least one member of a library of candidate polypeptides that may be GPCR polypeptides or non-GPCR polypeptides. Collectively, these polypeptides are termed “polypeptides of interest”, and the invention involves testing for binding between two polypeptides of interest, where one of the polypeptides of interest is a GPCR.

Polypeptides of interest include GPCRs. GPCRs are well known in the art, and, as described in the definitions section, are identifiable as having a common structural motif, i.e., seven sequences of between 22 to 24 hydrophobic amino acids that form seven alpha helices, each of which spans the membrane. Suitable GPCRs may be from any species that has a GPCR, and, as such GPCRs may be mammalian in origin (e.g., from humans, pig, rat, mouse, etc), or from any other species, such as D. melanogaster, C. elegans, A. thaliaia, etc. A disclosure of the sequences and phylogenetic relationships between 277 GPCRs is provided in Joost et al. (Genome Biol. 2002 3:RESEARCH0063, the entire contents of which is incorporated by reference) and, as such, at least 277 GPCRs are suitable for use the subject methods. A more recent disclosure of the sequences and phylogenetic relationships between 367 human and 392 mouse GPCRs is provided in Vassilatis et al. (Proc Natl Acad Sci 2003 100:4903-8), and a database of GPCRs may also be obtained from the world wide website of primalinc.com. The contents of Vassilatis et al and the Primalinc database are hereby incorporated by reference in their entirety.

Suitable GPCRs include, by way of exemplification, GPCRs that belong to families of GPCRs, such as the families of purinergic receptors, vitamin receptors, lipid receptors, peptide hormone receptors, non-hormone peptide receptors, non-peptide hormone receptors, polypeptide receptors, protease receptors, receptors for sensory signal mediator, and biogenic amine receptors.

Without any intention to limit the invention to any particular GPCR, the following is a list of particular GPCRs may be used in the subject methods: adrenergic receptor, beta-2 (“β2AR”); adrenergic receptor, beta-3 (“β3AR”); cholinergic receptor, muscarinic 1 (“M1”); cholinergic receptor, muscarinic 3 (“M3”); melanin-concentrating hormone receptor 2; cholinergic receptor, muscarinic 4; niacin receptor; histamine 4 receptor; ghrelin receptor (“GHSR”); hypocretin (orexin) receptor 1 (“OXR1”); GPR50; CXCR3 chemokine receptor; motilin receptor; 5-hydroxytryptamine (serotonin) receptor 2A (“5HT2A”); 5-hydroxytryptamine (serotonin) receptor 2B (“5HT2B”); 5-hydroxytryptamine (serotonin) receptor 2C (“5HT2C”); dopamine receptor D3; dopamine receptor D2 (“D2”); dopamine receptor D4; dopamine receptor D1 (“D1”); histamine receptor H2 (“H2”); histamine receptor H3 (“H3”); adrenergic receptor, alpha-2A (“α2AAR”); galanin receptor 1; neuropeptide Y receptor Y1 (“NPYR1”); neuropeptide Y receptor Y5 (“NPYR5”); angiotensin II receptor 1; neurotensin receptor 1; melanocortin 4 receptor (“MCR4”); glucagon-like peptide 1 receptor; adenosine A1 receptor (“ADORA1”); cannabinoid receptor 1; melanin-concentrating hormone receptor 1 (“MCHR1”); melatonin receptor 1B (“MTNR1B”); GPR40; and GPCR2. The amino acid and nucleotide sequences of these GPCRs may be found at the NCBI's Genbank database as accession numbers NM_{—}000024, NM_{—}000025, X15263, NM_{—}000740, AB058849, X15265, AB065876, NM_{—}021624, U60179, NM_{—}001525, NM_{—}004224, NM_{—}001504, NM_{—}001507, S71229, NM_{—}000867, NM_{—}000868, NM_{—}000796, S62137, L12398, X58987, NM_{—}022304, NM_{—}007232, J05652, NM_{—}001480, NM_{—}000909, BC034224, NM_{—}031850, NM_{—}002531, S77415, NM_{—}002062, NM_{—}000674, NM_{—}001840, NM_{—}005297, NM_{—}005959, NM_{—}005303 and XM_{—}066873.

Other polypeptides of interest include “GPCR accessory polypeptides”, where a GPCR accessory polypeptide is known or is thought to be involved in GPCR signaling. Exemplary GPCR accessory polypeptides include: receptor activity modifying proteins (i.e., “RAMPs”; reviewed in Sexton et al, (Cell. Sign. 13:73-83, 2001) and in Foord, (Matching Accessories, Science's STKE 2003: pe25), e.g., RAMP1, RAMP2 or RAMP3; the α, β or γ subunits of heterotrimeric G-proteins, e.g., the Gq, Gs, Gi, Gz, Go, G11, G12, G13, G15 and G16 α subunits; Homer family proteins (reviewed in Fagni et al, Homer as Both a Scaffold and Transduction Molecule, Science's STKE 2002: re8); regulators of G protein signaling (i.e., “RGSs”) and RGS-like proteins (reviewed in Hollinger et al, Pharmacol Rev 54:527-59, 2002); G protein-coupled receptor kinases (i.e., “GRKs”) (reviewed in Penn et al., Trends Cardiovasc Med 10:81-9, 2000); arrestins (reviewed in Perry et al., Trends Cell Biol 12:130-8, 2002); and small GTP binding proteins, e.g. Ras or Rho (reviewed in Manser, Dev Cell 3:323-8, 2002).

Other polypeptides of interest include any other polypeptide irrespective of whether the polypeptide is known or thought be to be involved in GPCR signaling, including polypeptides of known or unknown function, and polypeptides belonging to the following polypeptide families: kinases such as serine/threonine or tyrosine kinases (e.g., receptor tyrosine kinases; Agnati et al., Pharmacol Rev 55:509-550, 2003; the disclosure of which is hereby incorporated by reference in its entirety); the disclosure of which is hereby incorporated by reference in its entirety); DNA binding proteins such as transcription factors; peptide and non-peptide receptors; protein phosphatases; tetraspanins (reviewed in Stipp et al, Trends Biochem Sci 28:106-12, 2003); single-transmembrane growth hormone receptors; ion channel receptors; major histocompatibility complex (i.e. “MHC”) molecules, including HLA-DR and M10-related MHC molecules (reviewed in Foord, (Matching Accessories, Science's STKE 2003: pe25); adiponectin receptors AdipoR1 and AdipoR2 (Yamauchi et al, Nature 423:762-769, 2003) and splice variants thereof; other plasma membrane transmembrane proteins comprising at least one transmembrane domain; and any other polypeptide that contains a protein-protein interaction domain, e.g., an SH2, SH3 or PDZ domain, for example.

Polypeptide Variants

The term “polypeptides of interest” also includes variants of the above recited GPCR polypeptides, GPCR-accessory polypeptides, and other polypeptides. In other words, variants of any polypeptide may be used in the subject methods. In certain embodiments, therefore, a polypeptide of interest may have an altered sequence as compared to a native sequence (e.g., a sequence deposited in NCBI's Genbank database). For example, a polypeptide of interest may be a native polypeptide having any number of amino acid substitutions, amino acid deletions, or amino acid additions at any position in the polypeptide (e.g., the C- or N-terminus, or at internal positions). Such alterations in the amino acid sequence of the native polypeptide may alter the activity of the polypeptide, e.g., the amino acid alterations may make the polypeptide of interest a constitutively active, or inactive. For example, as is known in the art, a constitutively active GPCR may be made using a variety of methods. It is expressly contemplated that a constitutively activated variant of a native GPCR is within the scope of the subject invention (see, e.g., PCT Application Number PCT/US98/07496 published as WO 98/46995 on 22 Oct. 1998; U.S. Pat. No. 6,255,089; and U.S. Pat. No. 6,555,339; and the disclosure each of which is hereby incorporated by reference in its entirety).

In particular embodiments, a polypeptide of interest is a fusion protein, and may contain, for example, an affinity tag domain or a reporter domain. Suitable affinity tags include any amino acid sequence that may be specifically bound to another moiety, usually another polypeptide, most usually an antibody. Suitable affinity tags include epitope tags, for example, the V5 tag, the FLAG tag, the HA tag (from hemagglutinin influenza virus), the myc tag, and the like, as is known in the art. Suitable affinity tags also include domains for which, binding substrates are known, e.g., HIS, GST and MBP tags, as is known in the art, and domains from other proteins for which specific binding partners, e.g., antibodies, particularly monoclonal antibodies, are available. Suitable affinity tags also include any protein-protein interaction domain, such as a IgG Fc region, which may be specifically bound and detected using a suitable binding partner, e.g. the IgG Fc receptor. It is expressly contemplated that such a fusion protein may contain a heterologous N-terminal domain (e.g., an epitope tag) fused in-frame with a GPCR that has had its N-terminal methionine residue either deleted or substituted with an alternative amino acid. It is appreciated that a polypeptide of interest may first be made from a native polypeptide and then operably linked to a suitable reporter/tag as described above.

In some embodiments, a polypeptide of interest may be a fragment of a GPCR, wherein said GPCR fragment is biologically active.

Suitable reporter domains include any domain that can report the presence of a polypeptide. While it is recognized that an affinity tag may be used to report the presence of a polypeptide using, e.g., a labeled antibody that specifically binds to the tag, light emitting reporter domains are more usually used. Suitable light emitting reporter domains include luciferase (from, e.g., firefly, Vargula, Renilla reniformis or Renilla muelleri), or light emitting variants thereof. Other suitable reporter domains include fluorescent proteins, (from e.g., jellyfish, corals and other coelenterates as such those from Aequoria, Renilla, Ptilosarcus, Stylatula species), or light emitting variants thereof. Light emitting variants of these reporter proteins are very well known in the art and may be brighter, dimmer, or have different excitation and/or emission spectra, as compared to a native reporter protein. For example, some variants are altered such that they no longer appear green, and may appear blue, cyan, yellow, enhanced yellow red (termed BFP, CFP, YFP eYFP and RFP, respectively) or have other emission spectra, as is known in the art. Other suitable reporter domains include domains that can report the presence of a polypeptide through a biochemical or color change, such as β-galactosidase, β-glucuronidase, chloramphenicol acetyl transferase, and secreted embryonic alkaline phosphatase. In some preferred embodiments, the reporter domain is Renilla luciferase (e.g., pRLCMV; Promega, catalog number E2661).

Also as is known in the art, an affinity tags or a reporter domain may be present at any position in a polypeptide of interest. However, in most embodiments, they are present at the C- or N-terminal end of a polypeptide of interest.

In many embodiments, a polypeptide of interest is a member of a library of polypeptides of interest. Typically, a library contains a plurality of members, where a plurality may be 2 or more, 5 or more, about 10 or more, about 20 or more, about 50 or more, about 100 or more, about 200 or more, about 300 or more, about 500 or more, about 1000 or more, or even up to about 10,000 or more. The library may therefore contain about 5, about 10, about 20, about 30 or more, about 50 or more, about 100 or more, about 200 or more, usually up to 500 or more, usually up to about 1000 or more GPCR polypeptides. The members of the library may be of known identity, or unknown identity, or a mixture thereof.

Nucleic Acids Encoding Polypeptides of Interest

Since the genetic code and recombinant techniques for manipulating nucleic acid are known, and the amino acid sequences of polypeptides of interest described as above, the design and production of nucleic acids encoding a polypeptide of interest is well within the skill of an artisan. In certain embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) methods are used. For example, GPCR coding sequences may be isolated from a library of GPCR coding sequence using any one or a combination of a variety of recombinant methods that do not need to be described herein. Subsequent substitution, deletion, and/or addition of nucleotides in the nucleic acid sequence encoding a protein may also be done use standard recombinant DNA techniques.

For example, site directed mutagenesis and subcloning may be used to introduce/delete/substitute nucleic acid residues in a polynucleotide encoding a polypeptide of interest. In other embodiments, PCR may be used. Nucleic acids encoding a polypeptide of interest may also be made by chemical synthesis entirely from oligonucleotides (e.g., Cello et al., Science (2002) 297:1016-8).

In certain embodiments, the codons of the nucleic acids encoding polypeptides of interest are optimized for expression in cells of a particular species, particularly a mammalian, e.g., human, species.

The invention further provides vectors (also referred to as “constructs”) comprising a subject nucleic acid. In many embodiments of the invention, the subject nucleic acid sequences will be expressed in a host after the sequences have been operably linked to an expression control sequence, including, e.g. a promoter. The subject nucleic acids are also typically placed in an expression vector that can replicate in a host cell either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like. Retroviral, adenoviral and adeno-associated viral vectors may be used.

A variety of expression vectors are available to those in the art for purposes of producing a polypeptide of interest in a cell. One suitable vector is pCMV, which used in certain embodiments. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

The subject nucleic acids usually comprise an single open reading frame encoding a subject polypeptide of interest, however, in certain embodiments, since the host cell for expression of the polypeptide of interest may be a eukaryotic cell, e.g., a mammalian cell, such as a human cell, the open reading frame may be interrupted by introns. Subject nucleic acid are typically part of a transcriptional unit which may contain, in addition to the subject nucleic acid 3′ and 5′ untranslated regions (UTRs) which may direct RNA stability, translational efficiency, etc. The subject nucleic acid may also be part of an expression cassette which contains, in addition to the subject nucleic acid a promoter, which directs the transcription and expression of a polypeptide of interest, and a transcriptional terminator.

Eukaryotic promoters can be any promoter that is functional in a eukaryotic host cell, including viral promoters and promoters derived from eukaryotic genes. Exemplary eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a promoter of the target pathogen. Promoters for use in the present invention are selected such that they are functional in the cell type (and/or animal) into which they are being introduced. In certain embodiments, the promoter is a CMV promoter.

In certain embodiments, a subject vector may also provide for expression of a selectable marker. Suitable vectors and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001). Cold Spring Harbor, N.Y.). A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosphoribosyl transferase gene, CAD, the adenosine deaminase gene, the asparagine synthetase gene, the antibiotic resistance genes, e.g. tetr, ampr, Cmr or cat, kanr or neor (aminoglycoside phosphotransferase genes), the hygromycin B phosphotransferase gene, and the like.

As mentioned above, polypeptides of interest may be fusion proteins that contain an affinity domain and/or a reporter domain. Methods for making fusions between a reporter or tag and a GPCR, for example, at the C- or N-terminus of the GPCR, are well within the skill of one of skill in the art (e.g. McLean et al, Mol. Pharma. Mol Pharmacol. 1999 56:1182-91; Ramsay et al., Br. J. Pharmacology, 2001, 315-323) and will not be described any further. It is expressly contemplated that such a fusion protein may contain a heterologous N-terminal domain (e.g. an epitope tag) fused in-frame with a GPCR that has had its N-terminal methionine residue either deleted or substituted with an alternative amino acid. It is appreciated that a polypeptide of interest may first be made from a native polypeptide and then operably linked to a suitable reporter/tag as described above.

As mentioned above, a polypeptide of interest may be a fragment of a GPCR, wherein said GPCR fragment is biologically active.

The subject nucleic acids may also contain restriction sites, multiple cloning sites, primer binding sites, ligatable ends, recombination sites etc., usually in order to facilitate the construction of a nucleic acid encoding a polypeptide of interest.

Since a polypeptide of interest may be member of a library of polypeptides of interest, the nucleic acids encoding such a polypeptide of interest may also be a similar sized library of nucleic acids encoding polypeptides of interest.

GPCR-Binding Partner Complexes

The methods described herein involve, in many embodiments, detecting a “GPCR-binding partner complex” that contains at least one GPCR. By “GPCR-binding partner complex” is meant a complex of polypeptides that are associated with each other, either directly or indirectly, under “GPCR-binding partner binding conditions”.

GPCR-binding partner complexes include oligomers, where an oligomer may be a dimer, trimer, tetramer, or any other higher order oligomer of polypeptides of interest. Such oligomers may be a homooligomer (e.g., a homodimer), wherein each member of the complex is the same protein, or a heterooligomer (e.g., a heterodimer), in which the complex is made up of at least two different polypeptides of interest. Unless specifically set forth herein, the terms “dimer”, “trimer” or “tetramer” is not meant to exclude higher order oligomers. In other words, unless indicated to the contrary, the term “dimer” is intended to encompass a complex containing more than two polypeptides of interest. It is appreciated that two GPCRs in a complex may be derived from the same native GPCR and have identical or near identical sequence, except for a reporter or affinity domain. In these embodiments, a complex containing these two GPCRs would be termed a homodimer, even though the sequences of the GPCRs, because of the added domains, are different.

Host Cells

The methods described herein involve co-producing (i.e., producing in the same cell, regardless of the time at which they are produced), two polypeptides of interest. Suitable cells for producing the two polypeptides of interest include prokaryotic, e.g., bacterial cells, as well as eukaryotic cells e.g. an animal cell (for example an insect, mammal, fish, amphibian, bird or reptile cell), a plant cell (for example a maize or Arabidopsis cell), or a fungal cell (for example a S. cerevisiae cell). Any cell suitable for expression of two polypeptide of interest-encoding nucleic acid may be used as a host cell. Usually, an animal host cell line is used, examples of which are as follows: monkey kidney cells (COS cells), monkey kidney CVI cells transformed by SV40 (COS-7, ATCC CRL 165 1); human embryonic kidney cells (HEK-293, Graham et al. J. Gen Virol. 36:59 (1977)); HEK-293T cells; baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). In certain embodiments, melanophores are used. Melanophores are skin cells found in lower vertebrates. Relevant materials and methods will be followed according to the disclosure of U.S. Pat. No. 5,462,856 and U.S. Pat. No. 6,051,386. These patent disclosures are hereby incorporated by reference in their entirety. Additional cell lines will become apparent to those of ordinary skill in the art, and a wide variety of cell lines are available from the American Type Culture Collection, 10801 University Boulevard, Manassas, Va. 20110-2209.

Affinity Substrates

The methods described herein involve binding of a polypeptide of interest to an “affinity substrate”, i.e. a substrate that specifically binds the polypeptide of interest. An affinity substrate is contains a solid, semi-solid, or insoluble support and is made from any material appropriate for “capture”, i.e., binding, of a polypeptide of interest, and does not interfere with the detection method used. As will be appreciated by those in the art, the number of possible affinity substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers. In a preferred embodiment, the substrates allow optical detection and do not themselves appreciably fluoresce or emit light. In addition, as is known the art, the substrate may be coated with any number of materials, including polymers, such as dextrans, acrylamides, gelatins, agarose, biocompatible substances such as proteins including bovine and other mammalian serum albumin.

In most embodiments, the affinity substrate is coated in an agent that facilitates the specific binding (either directly or indirectly) of a polypeptide of interest to the substrate. For example, if a polypeptide of interest is to be bound by an antibody, the affinity substrate is usually coated with protein A, or some other agent, that binds an antibody. In another example, the substrate is coated in streptavidin, and can bind a biotinylated antibody with affinity to the polypeptide of interest. In another example, the substrate is directly or indirectly (e.g., through protein A) coated with a secondary antibody specific for the constant (e.g., Fc) region of a primary antibody that can bind with affinity to the polypeptide of interest.

A “spatially addressable” affinity substrate has multiple, discrete, regions (e.g., multiple polypeptide of interest-binding regions) such that each region is at a particular predetermined location (an “address”). Multi-well microtiter plates are addressable (each well having an address), an array of capillary columns is addressable, an array of samples deposited onto a solid support (e.g., a nylon or nitrocellulose membrane) is addressable. Affinity substrates for use in the methods described herein typically have at least 4 or more, at least about 12, at least about 24, at least about 48, at least about 96 or at least about 384 or addressable regions. In particular embodiments, an affinity substrate is in an addressable format suitable for high throughput assays, e.g., a 24-, 48- 96- or 384-well format, and each address of the substrate contains an agent (e.g., protein A or streptavidin or secondary antibody) for binding a polypeptide of interest (e.g., via an antibody that binds to protein A).

Such multi-well formats are suitable for use by robots, (e.g., pipetting robots), and other instrumentation (96- or 384-well format luminometers or fluorescence readers for determining reporter activity). By way of illustration and not limitation, reporter activity may be measured using a Wallac 1450 Microbeta counter (Perkin-Elmer) and in other embodiments, reporter activity measurements may involve a CCD camera-based illuminator.

In alternative embodiments, the addressable affinity substrate is a spectrophotometrically addressable affinity substrate. In one embodiment, the spectrophotometrically addressable affinity substrate is a microparticle with a unique spectrophotometric signature to which a particular capture antibody directed to a first polypeptide (e.g., a first GPCR) is conjugated. (The spectrophotometric signature may be provided by a particular ratio of two or more dyes.) It is envisioned that multiplexing may be carried out by assigning capture antibodies specific for different first polypeptides to beads having distinguishably different spectrophotometric signatures. By way of illustration and not limitation, one could in this manner interrogate simultaneously from one cell source binding of an epitope-tagged second polypeptide (e.g., a second GPCR) to a plurality (e.g., more than 2, more than 5, more than 10, more than 15, more than 20, or more than 25) of different first polypeptides (e.g., by using a phycoerythrin (PE)-conjugated antibody specific for the epitope tag). The LUMINEX™ platform (R&D systems, Minneapolis, Minn.) provides a suitable spectrophometrically addressable affinity substrate for use in the subject methods. The spectrophotometrically addressable format is less amenable to high-throughput (e.g., at least about 100 samples) than is the spatially addressable format, because of the limited number (about 15-25) of distinguishable spectrophotometric signatures.

Methods of Identifying a GPCR Complex

The invention provides a method for detecting a GPCR-binding partner complex. In general, the method involves three steps: a) co-producing two polypeptides of interest in a cell, one of which polypeptides of interest being a GPCR, b) “capturing” one of the polypeptides of interest (i.e., a “first” polypeptide of interest) using a substrate having affinity for that polypeptide under conditions suitable for binding of the polypeptide to the substrate; and, c) detecting the presence of the other polypeptide of interest (i.e., the “second” polypeptide of interest) on the substrate. If the other polypeptide of interest is detected, a GPCR-binding partner complex containing the two polypeptides is indicated: the two polypeptides are a GPCR and a GPCR-binding partner, which, as described above, may itself be a GPCR or any other protein. As such, the method, in addition to detecting a complex, provides a means by which a binding partner for a particular GPCR can be identified. In some embodiments, the affinity substrate is addressable, more particularly spatially addressable.

In some embodiments, particularly those in which the first and second polypeptides of interest are different, the polypeptides of interest are endogenously co-expressed by at least one cell type, tissue or tissue subregion. In other words, the polypeptides of interest may be co-expressed in a non-recombinant cell from a subject (e.g., a cell from any mammal or cultured cell thereof, etc.). Polypeptides expression may be measured directly, or may be measured indirectly, by assaying the level of mRNA encoding that polypeptide in a cell. Methods for determining expression of a polypeptide of interest, or an encoding nucleic acid thereof, are well known to those of skill in the art and include but are not limited to DNA chip, RT-PCR, Northern blot, in situ hybridization, immunohistochemistry, and flow cytometry. In particular embodiment, where said first and second polypeptide are different GPCRs, they are endogenously co-expressed by at least one cell type, tissue or tissue subregion.

As would be recognized by any skilled artisan, depending on how these assays are performed, either or both of the co-produced polypeptides may be a GPCR. In other words, either the first or the second or both the first and the second polypeptide of interest may be a GPCR.

In some embodiments, the subject methods may be performed with polypeptides of interest that do not contain a reporter or affinity domain. In these embodiments, the “capture” step may be done using an antibody that recognizes the first polypeptide but not the second polypeptide, and the detection step may be done using an antibody, e.g., a labeled antibody, that recognizes the second polypeptide but not the first polypeptide. In many embodiments, however, these methods are usually performed with fusion proteins, where the first polypeptide of interest is tagged with an affinity domain, and the second polypeptide of interest contains a reporter domain. The first polypeptide of interest may be captured using, for example, an antibody that binds to the affinity domain, and the second polypeptide of interest my be detected by measuring reporter activity, e.g., emission of a light. FIG. 1 shows a schematic representation of such an embodiment. In this embodiment, two GPCRs, GPCR A, having a luciferase reporter, and GPCR B, having a hemagglutinin epitope tag, are co-expressed a cell, and an extract of the cell is contacted with a protein A coated plate that has anti-hemagglutinin antibodies immobilized on its surface. After washing of the plate, detection of luciferase activity indicates that GPCR A and GPCR B form a complex, which, in this case, is a heterodimer.

In particular embodiments, the individual members of a library of polypeptides are systematically tested in pairwise combination to determine whether they may dimerize to form a GPCR-binding partner complex. In many embodiments, therefore, an addressable affinity substrate, at any one time, may be used to test binding between plurality of polypeptides of interest, e.g., a single GPCR and a plurality of candidate binding partners for that GPCR. In such embodiments, an address of the substrate serve to identify the polypeptides of interest that have been applied to that position of the substrate. For example, if polypeptide of interest “GPR6” and “GPR22” are added to well B6 of a 96-well affinity substrate, “B6” is the address, and, after capture, if a signal is detected from well B6, GPR6 and GPR22 are binding partners.

As will be further discussed below, the cell expressing a first and second polypeptides of interest are usually lysed prior to capture of any GPCR complexes, and, as such, capture and detection are usually performed using polypeptides of interest that are not present in an intact cell membrane (i.e., not in the membrane of an intact cell) at the time of capture and detection. For example, they may be present in a solubilized membrane fraction of a cell, not in a membrane of an intact cell.

Without any intention to limit the invention to any particular steps, and solely for ease of description of the invention, the following description is divided into three sections: co-production, capture, and detection.

Co-Production

As mentioned above, the subject methods involve co-producing (in other words, “co-expressing”) two polypeptides of interest in a cell. It is expressly contemplated that said co-producing may be either transient or stable.

In certain embodiments, a cell may endogenously produce both polypeptide of interest (i.e., they are non-recombinant polypeptides that are encoded and expressed by the unmodified genome of that cell). In other embodiments, a cell may produce only one of the polypeptides of interest endogenously. In these embodiments, the subject method may involve introducing an expression cassette for the other of the polypeptides of interest into a cell. In many embodiments, however, the subject methods involve introducing two expression cassettes, one for each of the polypeptides of interest, into a cell to facilitate the co-expression of the two polypeptides of interest in the cell. In general, methods for co-producing two polypeptides in a cell are well known in the art. For example, any method for producing the heavy and light chains of an antibody in a cell may be adapted to produce two polypeptides of interest in a cell. For example, a dual expression cassette vector, each expression cassette having polypeptide of interest-encoding sequences, may be introduced in a cell, or, more commonly, two different vectors, each containing a single expression cassette for a polypeptide of interest, may be introduced into the same cell.

As such, one or two vectors for production of two polypeptides of interest may be introduced into a cell. Methods of introducing vectors into cells are well known in the art. Suitable methods include electroporation, particle gun technology, calcium phosphate precipitation, direct microinjection, and the like. The choice of method is generally dependent on the vector being used, the type of cell being transformed and the circumstances under which the transformation is taking place. A general discussion of these methods can be found in Ausubel, et al, Short Protocols in Molecular Biology, 3rd ed., Wiley & Sons, 1995. In some embodiments lipofectamine and calcium mediated gene transfer technologies are used. Methods for introducing circular nucleic acids are also well known in the art and discussed in Ausubel, above. As is known in the art, this may be achieved by, for example, using a viral vector, e.g., a retroviral vector, and transfecting cell a sufficiently high titer of viral particles to introduce two different vectors into the cell.

After introduction of at least one expression cassette for polypeptide of interest into a cell, the cell is typically incubated to provide for polypeptide expression. To accomplish this, the cell may be incubated in suitable media for 12-24 hr, 24-48 hr, or 48-96 hr or more. Transient expression of the polypeptide may be carried out in this manner. It is expressly contemplated, however, that expression of the polypeptide may alternatively be stable. In said stable transfection, said expression cassette comprises a selectable marker gene and establishment of a stable cell line expressing the polypeptide comprises selection for the selectable marker gene. If two expression cassettes are introduced into a cell, the two expression cassettes may comprise different selectable marker genes (e.g. neomycin resistance gene and hygromycin resistance gene). Methods of transient and stable transfection are well known to those of skill in the art.

The two polypeptides of interest are usually pre-selected polypeptides of interest in that they are chosen and their identities are known prior to assaying whether or not they bind to each other. In certain embodiments, however, particularly if a library of polypeptides of interest is used the polypeptides of interest may be chosen at random, and their identities may not be known.

In certain embodiments, the polypeptides of interest are pre-selected based on prior knowledge. For example, two polypeptides of interest may be pre-selected of their co-expression or activity in normal cells (e.g., their simultaneous induction in response to a certain condition or treatment, their simultaneous expression in certain cells or tissues, or their simultaneous induction at a certain time of development), their binding to a common binding partner, or any other indication that the two polypeptides may bind together.

Capture

“Capture” refers to binding of a first polypeptide of interest to a substrate having affinity for that polypeptide. In general, capture involves lysing the cell producing the two polypeptides of interest, and contacting a cell extract, e.g., a whole cell lysate or a lysate of isolated membrane, with an addressable affinity substrate for the first polypeptide of interest. The cell extract is usually contacted with the addressable affinity substrate under conditions suitable for binding of the first polypeptide of interest to the affinity substrate, and the polypeptide binds to the substrate. In other words, the first polypeptide is captured by the affinity substrate. Since most embodiments of the invention involve specific binding of the first polypeptide of interest to the affinity substrate using an antibody (which antibody may be, e.g., immobilized on the protein A-coated substrate, or, in alternative embodiments a biotinylated antibody immobilized on a streptavidin-coated substrate), suitable conditions for such binding are well known in the art and are generally described in Harlow et al., (Antibodies: A Laboratory Manual, First Edition (1988) Cold Spring Harbor, N.Y.). Specific binding conditions may also including blocking steps and/or washing steps, as are known in the art.

If the first and second polypeptides of interest are be bound to each other in a complex, specific binding of the first polypeptide of interest to the affinity substrate serves to isolate, or, in other words, purify, the complex from other proteins in the cell extract. If no complexes form between the first and second polypeptides of interest, then only the first polypeptide will be bound to the affinity substrate, and the second polypeptide of interest will be washed from the substrate prior to detection.

As mentioned above, in most embodiments, cells are usually lysed to make a cell extract. Lysis is usually performed in the presence of a non-ionic detergent, e.g., Genapol C-100, Tween 20, Tween 40, Tween 65, Tween 80, Tween 85, Triton X-100, Lubrol PX, Nonidet P-40, Brij 35, SPAN, digitonin, or octyl-glucoside, etc.

Lysis and capture may be carried out within a range of salt concentration and pH, e.g. about 50-150 mM NaCl, typically about 50 mM NaCl, and about pH 7-8, typically about pH 7.4.

Detection

As mentioned above, after the first polypeptide of interest has been captured by the affinity substrate, the second polypeptide of interest is detectable on the affinity substrate only if the first and second polypeptides of interest form a complex. As such, in the subject methods, detection of the polypeptide of interest indicates that the first and second polypeptides form a complex. In preferred embodiments, said detecting of the presence of the second polypeptide on the affinity substrate is carried out directly (i.e., said detecting is “direct”), that is while the second polypeptide is bound to the affinity substrate (as opposed, e.g., to detection of said second polypeptide after elution from the affinity substrate).

In embodiments where the second polypeptide contains a reporter domain, the second polypeptide may be detected by detecting reporter activity. Methods of determining reporter activity, e.g. luciferase and GFP activity, are generally well known in the art (e.g. Ramsay et al., Br. J. Pharmacology, 2001, 133:315-323), and need not be described any further.

Detection of the second polypeptide may also be accomplished using an antibody, e.g., a labeled antibody. Methods for detecting polypeptides using antibodies are also well known in the art (e.g., Ausubel et al, Short Protocols in Molecular Biology, 3rd ed., Wiley & Sons, 1995; and Harlow et al., Antibodies: A Laboratory Manual, First Edition 1988 Cold Spring Harbor, N.Y.) and need not be described in an more detail.

In particularly embodiments, Fluorescence Resonance Energy Transfer (FRET) may be used to detect binding of two polypeptides of interest to form a complex. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (See, U.S. Pat. No. 5,981,200, the disclosure of which is hereby incorporated by reference in its entirety.)

For example, a cyan fluorescent protein is excited by light at roughly 425-450 nm wavelength and emits light in the range of 450-500 nm. Yellow fluorescent protein is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor:acceptor emission ratio is low, FRET is occurring and the two fluorescent proteins are in close proximity. In this manner, the interaction between a first and second polypeptide fused to a first and second reactive module, wherein the first and second reactive modules are donor and acceptor fluorescent molecules, respectively, may be measured. As such, the two polypeptides of interest may contain a system that provides for FRET, e.g., one polypeptide contains GFP whereas the other contains YFP.

In a further embodiment, the first and second polypeptides of interest provide a Bioluminescence Resonance Energy Transfer (BRET) system. In such a system, one polypeptide of interest produces (or destroys) a fluorescent product (or substrate) and the other polypeptide of interest is a fluorescent protein that undergoes resonant energy transfer with the fluorescent product (or substrate). In one embodiment, a BRET system comprises a luciferase from Renilla and a GFP. Exemplary BRET methodologies are described in Kroeger et al., J Biol Chem. 2001 Apr. 20; 276(16):12736-43 and Xu et al., Proc Natl Acad Sci USA. 1999 Jan. 5; 96(1):151-6.

In certain embodiments, a cross-linking agent may be used in the above methods of identifying a GPCR complex, wherein a cross-linking agent may be a homo- or heterobifunctional linker having a group at one end capable of forming a stable linkage to a first polypeptide of interest, and a group at the opposite end capable of forming a stable linkage to the second polypeptide of interest. Illustrative entities include: azidobenzoyl hydrazide, N [4(p azidosalicylamino)butyl]-3′[2′ pyridyldithio]propionamide), bis-sulfosuccinimidyl suberate, dimethyladipimidate, disuccinimidyltartrate, N g maleimidobutyryloxysuccinimide ester, N-hydroxy sulfosuccinimidyl-4-azidobenzoate, N succinimidyl [4 azidophenyl]-1,3′-dithiopropionate, N succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N,N′-(1,3-phenylene)bismaleimide; N,N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Homobifunctional cross-linking reagents include bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain, and are useful for linking polypeptides that contain cysteine residues.

In particular, homobifunctional cross-lining agents with reactive amine groups may be used since most proteins of interest contain a free N-terminal amino and limited lysine residues with ε-amine groups. Bis(sulfosuccinimidyl) suberate (BS), disuccinimidyl suberate (DSS), dithiobis(succinimidyl propionate) (DSP), 3,3′-dithiobis(sulfosuccinimidyl propionate) (DTSSP), and sulfosuccinimidyl 6-(biotinamido)hexanoate may be used.

For cross-linking polypeptides on the surface of a cell, membrane impermeable cross-linkers may be used.

In general, cells co-producing polypeptides of interest, or membrane preparation thereof are incubated with a cross-linking agent in a reaction buffer for a period of time prior to treatment with any detergent. After cross-linking, the cells or membrane are treated with a detergent, as described above, and binding between two polypeptides of interest is detected. Guidance for performing these cross-linking studies may also be found in Table 1 of Rios et al. Pharmacol Ther. 2001 92:71-87.

The subject invention therefore provide methods by which GPCR interactions may be identified. In some embodiments, therefore, a cell producing a GPCR may be lysed, and the GPCR captured by a solid support. The binding partners for that GPCR may be identified using the methods set forth herein, or by other biochemical or physical methods known in the art (e.g., mass spectrometry, protein electrophoresis, gas or liquid chromatography, etc.). In particular embodiments, the “natural” ligand may be identified using these methods. In these embodiments, detecting alterations of the components of a GPCR complex in the presence or absence of a particular binding partner of a GPCR are of particular interest. For example, the methods may be performed using a cell producing a GPCR and another cell producing a GPCR and a binding partner for that GPCR. The subject methods may be performed on each of the cells, and the components of the GPCR complexes for each of the complexes may be identified. These methods find particular use in understanding GPCR complex mediated signal transduction.

Further, the subject methods provide a means by which ligands for a GPCR may be identified. In these embodiments, a cell producing a GPCR, with or without a binding partner for the GPCR, may be contacted with a candidate ligand for the GPCR (e.g., a peptide, cell fractions that are proteinaceous or non-proteinaceous, chemical libraries, a random peptide library etc.), the cell is lysed, and the GPCR is captured; as discussed above. The moieties bound to the GPCR may be identified using the methods set forth herein, or by other biochemical or physical methods known in the art (e.g., mass spectrometry, protein electrophoresis, gas or liquid chromatography, etc.).

Method of Identifying a Modulator of Binding Between the Polypeptides of a GPCR-Binding Partner Complex

The invention also provides a means by which agents that modulate binding of a particular GPCR and a binding partner for that GPCR in a GPCR-binding partner complex may be identified. By “modulate binding” is meant inhibiting binding, e.g., inhibiting formation of the complex, disrupting the complex after it has formed, decreasing the strength of binding between a GPCR and a binding partner for the GPCR, etc., or increasing binding between members of the complex, e.g., increasing the strength of binding between a GPCR and a binding partner for the GPCR, promoting the formation of the complex, etc.

In general, the methods involve identifying a GPCR and a binding partner for that GPCR using the methods described in the preceding section, and assessing a candidate agent for an activity that modulates binding of that GPCR and its binding partner. In a first set of embodiments, these methods involve contacting a cell expressing at least two polypeptides of interest with a candidate agent for an interval of time prior to lysis. In a second set of embodiments, these methods involve contacting a GPCR-binding partner complex with a candidate agent, and determining the effects of the agent on the complex. The agent may be added to a cell for producing the first and second polypeptides of interest prior to or during production of at least one of the polypeptides, or, in other embodiments, the agent may be contacted with the complex during or after cell lysis, e.g., while any GPCR-binding partner complex is captured by the affinity substrate. In the aforementioned first and second sets of embodiments, the level of binding between a GPCR and a binding partner for that GPCR in a GPCR-binding partner complex is usually determined by again measuring the level of the second GPCR in the complex. For example, if a candidate agent is added to cells co-producing two polypeptides of interest, the cells are usually lysed, and the first polypeptide of interest is captured using an affinity substrate. The level of second polypeptide also captured (i.e., captured indirectly by virtue of it binding to the first polypeptide of interest) by the affinity substrate is determined using the methods described above (e.g., measuring reporter activity), and compared to suitable controls, which, in these embodiments, may be from experiments performed in the same manner, in the absence of a test agent, in the presence of an agent of no effect. If a candidate agent is added to a GPCR-binding partner complex after the complex is captured by the affinity matrix, again, the level of binding between the components of the complex may be determined by assessing the presence of the second polypeptide of interest, by, e.g. reporter activity. In these embodiments, the level of the second polypeptide of interest may be assessed in the absence of the agent, the complex contacted with the agent, and, after a period of time and any necessary washes, the level of the second polypeptide of interest is determined. Suitable controls for these assays also include assaying the second polypeptide of interest to determine if any reporter activity of that polypeptide is affected by the agent.

Any agent that reduces the level of the second polypeptide of interest in a GPCR-binding partner complex is an inhibitor of binding of that GPCR and its binding partner, and, accordingly, any agent that increases the level of the second polypeptide of interest in a GPCR-binding partner complex increases binding of that GPCR and its binding partner.

In general, if an agent is an inhibitor of binding of a GPCR and its binding partner, the level of the second binding partner in a GPCR-binding partner complex captured on an affinity substrate, as determined by a reporter activity or, e.g., its binding to an antibody, is usually reduced by greater than about 10%, greater than about 20%, greater than about 30%, greater than about 40%, greater than about 50%, greater than about 60%, greater than about 80%, greater than about 90%, greater than about 95%, greater than about 98%, or greater than about 99%, as compared to suitable controls.

If an agent is an increases binding of GPCR and its binding partner, the level of the second binding partner in a GPCR-binding partner complex captured on an affinity substrate, as determined by a reporter activity or, e.g., its binding to an antibody, is usually increased by greater than about 10%, greater than about 25%, greater than about 50%, greater than about 80%, greater than about 100%, greater than about 150%, greater than about 200%, greater than about 300%, greater than about 400%, greater than about 500%, or greater than about 1,000%, as compared to suitable controls.

In certain embodiments, these assays may be performed in the presence of a ligand for at least one of the GPCRs in the GPCR complex, and, in other embodiments, the effects of a ligand on an activity of a GPCR, while is complexed with a binding partner for the GPCR, may also be determined.

In other embodiments, the subject screening methods may be performed using a cross-linking agent, such as a cross-linking agent recited in the previous section. In these embodiments, the cross-linker may be added at any time of the method. In most embodiments the cross-linking agent is usually added after incubation of cells with a test compound and prior to addition of non-ionic detergent (i.e., prior to cells containing the polypeptides of interest).

A variety of different test compounds may be screened using the above methods. Test compounds encompass numerous chemical classes, though typically they are organic molecules, preferably small organic compounds having a molecular weight of more than 50 and less than about 2,500 daltons. Test compounds comprise functional groups necessary for structural interaction with proteins, particularly hydrogen bonding, and typically include at least an amine, carbonyl, hydroxyl or carboxyl group, preferably at least two of the functional chemical groups. The test compounds often comprise cyclical carbon or heterocyclic structures and/or aromatic or polyaromatic structures substituted with one or more of the above functional groups. Test compounds are also found among biomolecules including peptides, saccharides, fatty acids, steroids, purines, pyrimidines, derivatives, structural analogs or combinations thereof. Further test compounds include variants of the GPCR's native ligand.

Test compounds may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Of interest are test compounds that are polypeptides, e.g., proteinaceous, agents. A specific type of polypeptide test compound of interest is an antibody for the GPCR, or a GPCR-binding fragment thereof. The antibody may be monoclonal or polyclonal, and may be produced according to methods known in the art. Further test compounds include variants of the GPCR's native ligand, e.g. a native ligand that is altered by substitution, deletion or addition of at least one amino acid, or chemically modified. In certain embodiments test compounds include endogenous polypeptides not known to be ligands of the GPCR.

Methods of Modulating Activity of a GPCR-Binding Partner Complex

Once identified, a binding partner for a GPCR may be tested for an ability to modulate the activity of that GPCR. As such, the invention provides methods of modulating GPCR activity, where the GPCR is a component of a GPCR-binding partner complex. In many respects, these assays are similar to traditional GPCR activity assays, except the GPCR is present as part of a GPCR-binding partner complex. In other words, an activity of a GPCR in a GPCR-binding partner complex may be determined, and compared to the activity of that GPCR alone (i.e., that GPCR, but not in a GPCR-binding partner complex).

In certain embodiments, therefore, these assays involve producing a GPCR in a cell, and determining the effect of co-production of a binding partner for that GPCR on an activity of that GPCR. As is known in the art, several such assays may be performed, such as, for example, membrane binding assays using ³⁵S GTPγS, adenylyl cyclase assays (e.g., using the FLASH PLATE™ Adenylyl Cyclase kit from New England Nuclear; Cat. No. SMP004A), cell-based cAMP assays, reporter-based assays, AP1 reporter assays, SRF-LUC reporter assays, intracellular IP₃ accumulation assay, fluorometric imaging plate reader (FLIPR) assays for the measurement of intracellular calcium concentration, and melanophore assay (see, e.g., PCT patent publication no. WO 02/068600, the disclosure of which is hereby incorporated by reference in its entirety).

In other words, an modulator of a GPCR (e.g. a ligand for that GPCR, for example) may be contacted with a GPCR-binding partner complex containing that GPCR, and the effect of the modulator on the activity of the modulator on an activity of the GPCR complex may be assessed. Also envisioned are assays that are done using two modulators of a GPCR, e.g., an activator of a GPCR (for example, a ligand for that GPCR), and an agent that blocks the modulatory activity of the activator.

Utility

The subject methods for identifying GPCR-binding partner complexes find use in a variety of research and therapeutic protocols.

For example, the methods could be used to understand which receptors in any one cell type heterodimerize. In some embodiments, expression data (e.g., data from microarray experiments) provides information as to which receptors are endogenously coexpressed in at least one cell type, tissue or tissue sub-region. Expression vectors are made of these receptors and receptor dimerization is measured. For example, if receptors A, B, C, D, E are endogenously coexpressed in adipocytes, two constructs could be made for each receptor, one construct encoding an HA-tagged receptor and the other construct encoding a luciferase tagged receptor. Using the methods described above, HA-tagged receptor A is captured and luciferase tagged receptors A, B, C, D and E are tested for dimerization efficiency.

Further, the methods could be used to assess the level binding of a GPCR to a binding partner for that GPCR in the presence of known ligands, such as inverse agonists, agonists or antagonists for that GPCR. In addition, the methods could be used to screen compound libraries for compounds that enhance or inhibit the binding of a GPCR to a binding partner for that GPCR. The compound libraries could be small molecule libraries for example or natural product libraries.

Further, the methods could be used to measure GPCR accessory protein interaction with particular GPCRs e.g. RAMPs, and further, the assays find use in measuring receptor homodimerization and heterodimerization.

In particular, however, the subject methods find most use in b) identifying novel cellular targets (i.e., GPCR-binding partner complexes) that may be investigated as targets for pharmaceutical drugs, and in b) identifying pharmaceutical compositions for treatment of GPCR-binding partner complex-related conditions.

In other words, once a GPCR-binding partner complex have been identified, agents may be developed that modulate an activity of the complex by modulating the binding of a GPCR and a GPCR-binding partner in the complex, or by modulating selectively the activity of the GPCR-binding partner complex. Once discovered, these agents may be administered to an individual suffering from a GPCR-binding partner complex-related disorder in an effective amount to treat the individual for the disorder.

It is further noted that once a binding partner has been identified, if the pharmacology of a GPCR can be shown to be altered in the presence of the binding partner, then it follows that the pharmacology of that GPCR on one tissue may differ from the pharmacology of that GPCR on another tissue if the tissues differ in expression of the binding partner. This is expressly contemplated as facilitating the systematic and rational development of drugs that selectively modulate that GPCR in the desired tissue, thereby circumventing undesirable side effects through action on that GPCR at other than the desired tissue.

In some embodiments, where a reduction in activity of a certain GPCR-binding partner complex is desired, one or more compounds that decrease the activity of the GPCR-binding partner complex may be administered, whereas when an increase in activity of a certain GPCR-binding partner complex is desired, one or more compounds that increase the activity of the GPCR-binding partner complex may be administered.

A variety of individuals are treatable according to the subject methods. Generally such individuals are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the individuals will be humans.

Subject treatment methods are typically performed on individuals with such disorders or on individuals with a desire to avoid contracting such disorders. The invention also includes preventing or reducing the risk of a GPCR-binding partner complex-related condition by administering a pharmaceutical composition comprising a modulator selective for the GPCR complexes.

Kits

Also provided by the subject invention are kits for practicing the subject methods, as described above. The subject kits at least include one or more of: nucleic acids encoding at least two polypeptides of interest, one of which being a GPCR, and an affinity substrate for that GPCR. The nucleic acids of the kit may also have restrictions sites, multiple cloning sites, primer sites, etc to facilitate their ligation other plasmids. Other optional components of the kit include: a library of polypeptide of interest-encoding nucleic acids, nucleic acids encoding affinity or reporter domains, other components described above, and buffers, cells etc for performing the subject assays. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the subject invention.

### Materials and Methods

Reagents: Anti-FLAG antibody (monoclonal M2; Sigma, catalog number F-3165), Protein A (Sigma, catalog number P-3838). Coelenterazine (Biotum, catalog number 10110). anti-HA (12CA5) (Roche Molecular Biochemicals, catalog number 1 666 606). Anti-Gαs Santa Cruz biotechnology, catalog number sc-383). pRLCMV (Promega, catalog number E2661), pCDNA3.1 (+) (Invitrogen, catalog number V790-20).

Construction of Fusion Receptor Genes:

(1) HA-β₂AR—A full length cDNA encoding human β₂AR without Met, generated by PCR, was subcloned in-frame into HA-pCMV vector (pCMV vector modified through insertion of an HA epitope tag upstream of the cDNA cloning site) with Hind III and Xba I sites.

(2) FLAG-β₂AR—A full length cDNA encoding human β₂AR without Met, generated by PCR, was subcloned in-frame into FLAG-pCMV vector (pCMV vector modified through insertion of a FLAG epitope tag upstream of the cDNA cloning site).

(3) β₂AR-Rlu. First, a full-length cDNA encoding Renilla luciferase (Rlu; 312 amino acids), generated by PCR amplification of a Renilla luciferase-containing vector plasmid pRLCMV, was digested with EcoR I and Xba I, and 1 kb EcoR I-Xba I fragment was subcloned into pCDNA3.1 (+) vector, resulting in Rlu-pCDNA3.1. In the second step, a gene coding a full length human β₂AR without the stop code was generated by PCR amplification human β₂AR-pCMV plasmid. The PCR product, after digested with Hind III and EcoR I, was inserted into Rlu-pCDNA3.1 by Hind III and EcoR I, resulting in final construct β₂AR-Rlu with β₂AR upstream and in-frame with Rlu. Introduction of EcoR I restriction site between C-terminus of β₂AR and N-terminus of Rlu resulted in insertion of three additional amino acids link Glu-Asn-Ser.

(4) 5HT_{2C}-Rlu—First, a full-length cDNA encoding Renilla luciferase (Rlu; 312 amino acids), generated by PCR amplification of a Renilla luciferase-containing vector plasmid pRLCMV, was digested with EcoR I and Xba I, and 1 kb EcoR I-Xba I fragment was subcloned into pCDNA3.1 (+) vector, resulting in Rlu-pCDNA3.1. In the second step, a gene coding a full-length human 5HT_{2C} without the stop code was generated by PCR amplification human 5HT_{2C}-pCMV plasmid. The PCR product, after digested with Nhe I and EcoR I, was inserted into Rlu-pCDNA3.1 by Nhe I and EcoR I, resulting in final construct 5HT_{2C}-Rlu β₂AR upstream and in-frame with Rlu. Introduction of EcoR I restriction site between C-terminus of 5HT_{2C} and N-terminus of Rlu resulted in insertion of three additional amino acids link Glu-Asn-Ser.

Cell culture and transient transfection: HEK293 cells were transfected with receptor plasmids (normally 1:1 ratio for receptor cotransfection, 4 μg total plasmids per 100 cm dish) by LipofectAmine. 40 h after transfection cells were washed once with cold PBS and harvested in buffer A (20 mM Hepes, pH 7.5, 50 mM NaCl, 1 mM EDTA). Cell pellet was freshly used or stored at −80° C.

Generation of stable cell lines recombinantly expressing a GPCR and a binding partner for that GPCR: By way of illustration and not limitation, details are provided here for the case wherein the binding partner is a different GPCR (“GPCR A” and “GPCR B”). Generally, affinity-tagged [e.g., HA(FLAG)-tagged] GPCR A was constructed in an expression vector containing neomycin selectable marker gene, and GPCR B fused to a reporter gene (e.g., Renilla luciferase) was constructed in an expression vector containing hygromycin selectable marker gene. HEK 293 or CHO cells were grown in Dulbecco's modified Eagle's medium (DMEM) supplemented with 10% fetal calf serum, 2 mM L-glutamine, 100 μg/ml penicillin and 100 units/ml streptomycin at 37° C. in a humidified 5% CO₂ incubator. Cells (1×10⁶) were seeded into 100 mm dishes and after incubation for 24 h the cells were co-transfected with 4 μg of plasmid DNAs/dish (GPCR A and GPCR B at ratio of 1:1 (w/w) h using LipofectAmine™ reagent according to the manufacturer's instructions (Invitrogen). Two days after transfection, cells were split and seeded in 96-well plates and maintained in DMEM supplemented with 500 μg/ml G-418 and 100 μg/ml of hygromycin B. The medium was replaced every three days with DMEM supplemented with both antibiotics. Stable clones expressing two GPCRs were selected by FACS to confirm expression of the affinity-tagged [e.g., HA(FLAG)-tagged] GPCR A and by measuring reporter gene activity (e.g., Relative Light Units) to verify GPCR B expression.

Preparation of cell lysate: Cell pellets were resuspended in buffer A supplemented with protease inhibitors (1 mM PMSF, 1 μg/ml pepstatin A and 10 μg/ml leupeptin) and homogenized with a polytron. After centrifugation at 45,000 g for 15 min at 4° C., the supernatant was removed. The pellet was resuspended in lysis buffer (buffer A+detergent+protease inhibitors) (detergents were non-ionic detergents like digitonin, Triton X-100, NP-40, and concentration as indicated in Figures. Normally we used 0.5-1% digitonin for our experiments if no indicated) and kept in a rotator at 4° C. for 30-60 min. The supernatant was collected after centrifugation at 100,000 g at 4° C. for 30 min. Protein concentration was determined by method of Bradford (Bradford MM (1976) Anal Biochem. 72: 248-254).

Determination of receptor expression: To measure total receptor expression, 5-10 μg of lysate protein was mixed with 100 μl of 2 μM coelenterazine in buffer B (0.1 M sodium phosphate, pH 7.4, 0.5 M NaCl) in 96-well white plate. Luciferase activity was immediately measured with a Wallac Microbeta counter.

ELISA-based assay to determine receptor interaction: 96-well ELISA plate (white, high protein binding) was coated with protein A (100 μl/well, 5 μg/ml in 0.1 M NaHCO₃ at 4° C. overnight. The plates then washed four times with PBS and blocked with 1% BSA in PBS at RT for 1 h. After washing with wash buffer (PBS+0.05% Tween 20), the cell lysate (in 50 μl buffer A containing indicated total protein amount and concentration of detergent) was added into each well and then 50 μl of antibody solution (anti-HA, anti-FLAG or anti-V5: 5 μg/ml in PBS containing 1% BSA. The plate was incubated with shaking at RT for 1 h, then washed with wash buffer six times. 100 μl of 2 μM coelenterazine in buffer B (0.1 M sodium phosphate, pH 7.4, 0.5 M NaCl) was added. Luciferase activity was immediately measured with a Wallac Microbeta counter.

Data analysis: Homodimer formation of FLAG-beta2-adrenergic receptor (FLAG-β₂AR) contransfected with C-terminally Renilla luciferase tagged beta2-adrenergic receptor (β₂AR-Rlu) was set as 100%. Data for other receptors were normalized based on FLAG-β₂AR/(β₂AR-Rlu.

### Example 1

### Detection of β₂AR Dimer as Anti-FLAG Antibody-Bound β₂AR-Rlu

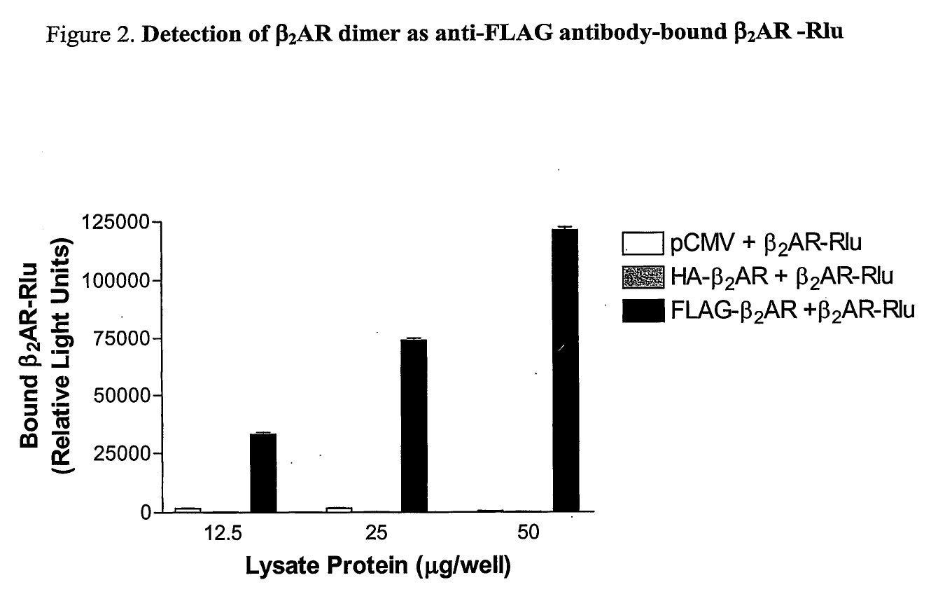

HEK293 cells were transiently transfected with β₂AR-Rlu and pCMV, HA-β₂AR or FLAG-β₂AR at ratio of 1:1 (w/w). Cells were harvested after about 40 h transfection and lysed as described under “Experimental methods”. To protein A-coated binding plate 50 μl of cell lysate containing indicated total protein amount and 0.25% digitonin was added, then 50 μl of anti-FLAG antibody solution (5 μg/ml in PBS containing 1% BSA) was added. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Dimeric β₂AR formation is expressed as luciferase activity of bound β₂AR-Rlu and represent one of three independent experiments, performed in triplicate. The results of these assays are shown in FIG. 2. The results show that specific FLAG-β₂AR/β₂AR-Rlu dimer was detected only by anti-FLAG antibody in receptor amount-dependent manner.

### Example 2

### Comparison of Anti-HA Antibody and Anti-FLAG Antibody in Detection of β₂AR Dimer

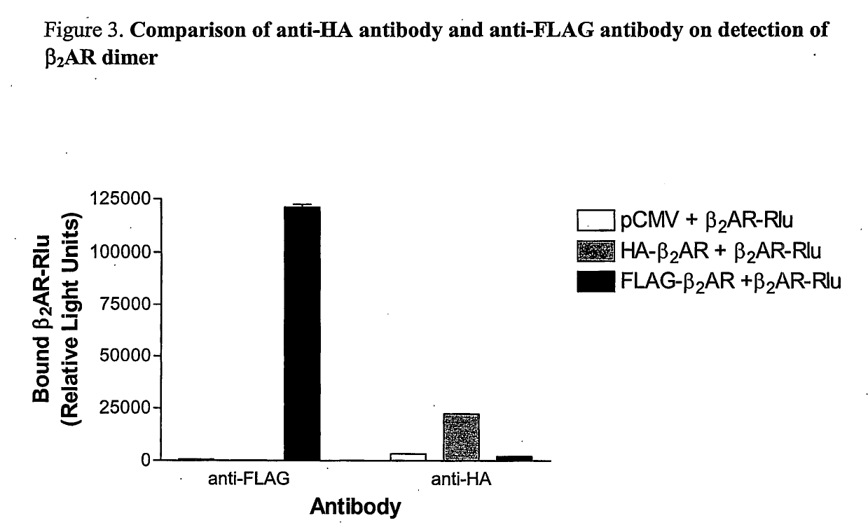

HEK293 cells were transiently transfected with β₂AR-Rlu and pCMV, HA-β₂AR or FLAG-β₂AR at ratio of 1:1 (w/w). Cells were harvested after about 40 h transfection and lysed as described under “Experimental methods”. To protein A-coated binding plate 50 μl of cell lysate containing 50 μg total proteins and 0.25% digitonin was added, then 50 μl of antibody solution (anti-HA or anti-FLAG: 5 μg/ml in PBS containing 1% BSA) was added. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Dimeric β₂AR formation is expressed as luciferase activity of bound β₂AR-Rlu and represent one of three independent experiments, performed in triplicate. The results of these assays are shown in FIG. 3. The results show that anti-FLAG and anti_HA antibodies were useful for detecting β₂AR dimer, although the anti-FLAG antibody showed better specificity and higher sensitivity than anti-HA.

### Example 3

### Effect of Digitonin Concentration on Detection of β₂AR Dimer

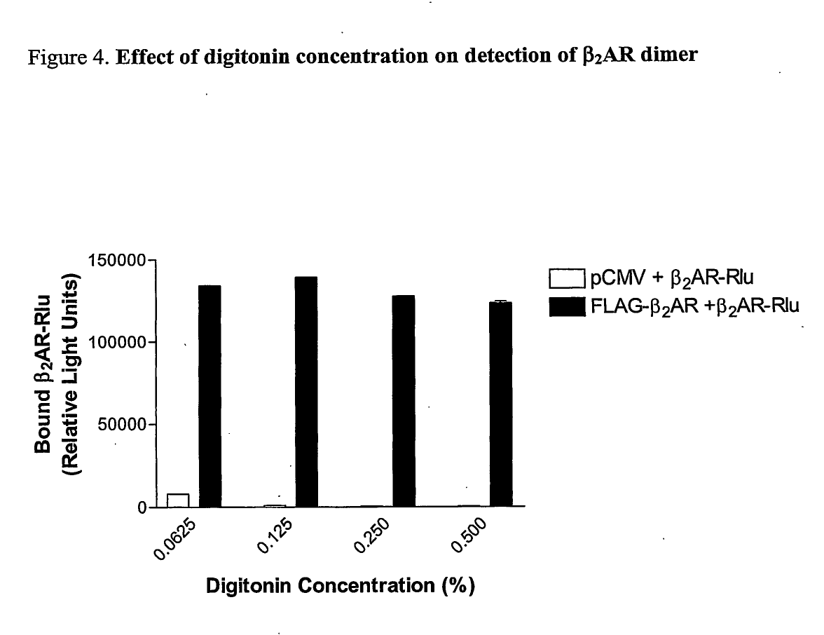

HEK293 cells were transiently transfected with β₂AR-Rlu and pCMV or FLAG-β₂AR at ratio of 1:1 (w/w). Cells were harvested after about 40 h transfection and lysed as described under “Experimental methods”. To protein A-coated binding plate 50 μl of cell lysate containing 12.5 μg total proteins and indicated concentrations of digitonin was added, then 50 μl of anti-FLAG antibody solution (5 μg/ml in PBS containing 1% BSA) was added. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Dimeric β₂AR formation is expressed as luciferase activity of bound β₂AR-Rlu and represent one of three independent experiments, performed in triplicate. The results of these assays are shown in FIG. 4. The results show that digitonin concentration, up to 0.5%, has no significant effect on stability of FLAG-ββ₂AR/β₂AR-Rlu dimer.

### Example 4

### Comparison of Digitonin and Triton X-100 for Solubilization and Detection of β₂AR Dimer

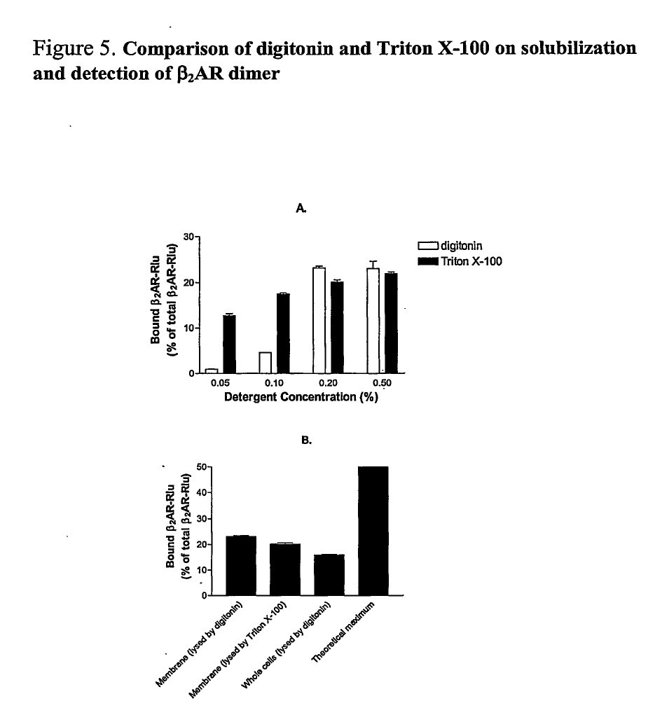

(A) Effect of detergent concentration. HEK293 cells were transiently transfected with β₂AR-Rlu and FLAG-β₂AR at ratio of 1:1 (w/w). Cells were harvested after about 40 h transfection and lysed with various concentrations of digitonin or Triton X-100 as described under “Experimental methods”. To protein A-coated binding plate 50 μl of cell lysate containing 12.5 μg total proteins and indicated concentrations of detergent was added, then 50 μl of anti-FLAG antibody solution (5 μg/ml in PBS containing 1% BSA) was added. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Dimeric β₂AR formation is expressed as percentage of total β₂AR-Rlu with a theoretical maximum of 50%. Data represent one of three independent experiments, performed in triplicate. The results of these assays are shown in FIG. 5, panel A. These results shows that both digitonin (>0.2%) and Triton X-100 (>0.1%) efficiently solubilized β₂AR receptors from membranes.

(B) Effect of lysate preparation procedures on detection of β₂AR dimer. HEK293 cells were transiently transfected with β₂AR-Rlu and FLAG-β₂AR at ratio of 1:1 (w/w). Cells were harvested after about 40 h transfection. Whole cells or cell membranes were lysed with 0.5% of digitonin or Triton X-100 as described under “experimental methods”. To protein A-coated binding plate 50 μl of cell lysate containing 12.5 μg total proteins and 0.125% of detergent was added, then 50 μl of anti-FLAG antibody solution (5 μg/ml in PBS containing 1% BSA) was added. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Dimeric β₂AR formation is expressed as percentage of total β₂AR-Rlu with a theoretical maximum of 50%. Data represent one of three independent experiments, performed in triplicate. The results of these assays are shown in FIG. 5, panel B. These results show that under optimal, maximal conditions (FLAG-β₂AR and β₂AR-Rlu expressed at 1:1 ratio), 50% of β₂AR-Rlu formed dimer with FLAG-β₂AR. Detected FLAG-β₂AR and β₂AR-Rlu dimer formation was in the range of 18-25%.

### Example 5

### Detection of β₂AR Dimer Forms in Cells, not Due to Non-Specific Protein Interaction During Experimental Procedures

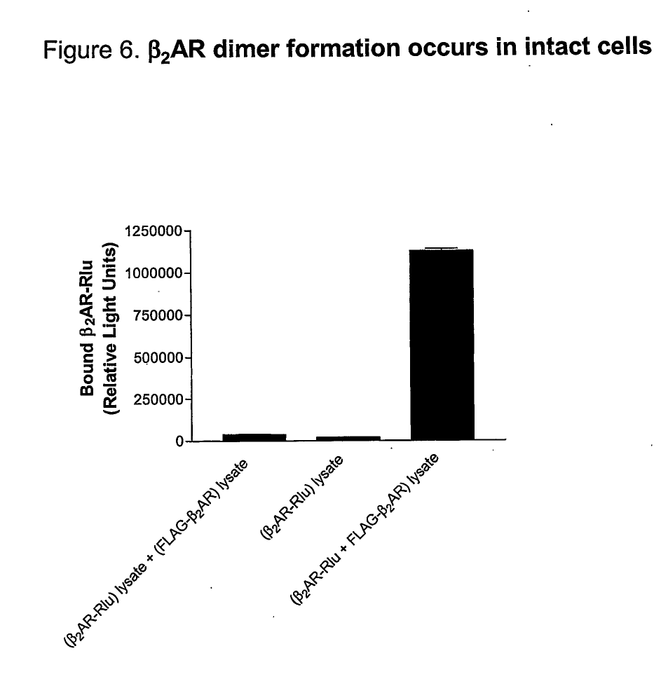

HEK₉₃ cells were transiently transfected with (A) β₂AR-Rlu+pCMV, (B) FLAG-β₂AR+pCMV or (C) β₂AR-Rlu+FLAG-β₂AR. Cell lysates were prepared as described under “Experimental methods”. A total of 50 μl of cell lyaste containing 12.5 μg of (A) and 12.5 μg of (3), 25 μg of (A) or 25 μg of (C) was used for assay. Dimeric β₂AR formation is expressed as luciferase activity of bound β₂AR-Rlu and represent one of three independent experiments, performed in triplicate. The results of these assays are shown in FIG. 6. These results show that dimeric FLAG-β₂AR/β₂AR-Rlu could only be detected when FLAG-β₂AR and β₂AR-Rlu were cotransfected. No significant dimeric FLAG-β₂AR/β₂AR-Rlu could be detected when lysates from FLAG-β₂AR-expressing cells and β₂AR-Rlu-expressing cells. These results indicate that β₂AR dimerization occurs in intact cells.

### Example 6

### Heterodimerization of β₂AR with other GPCRs

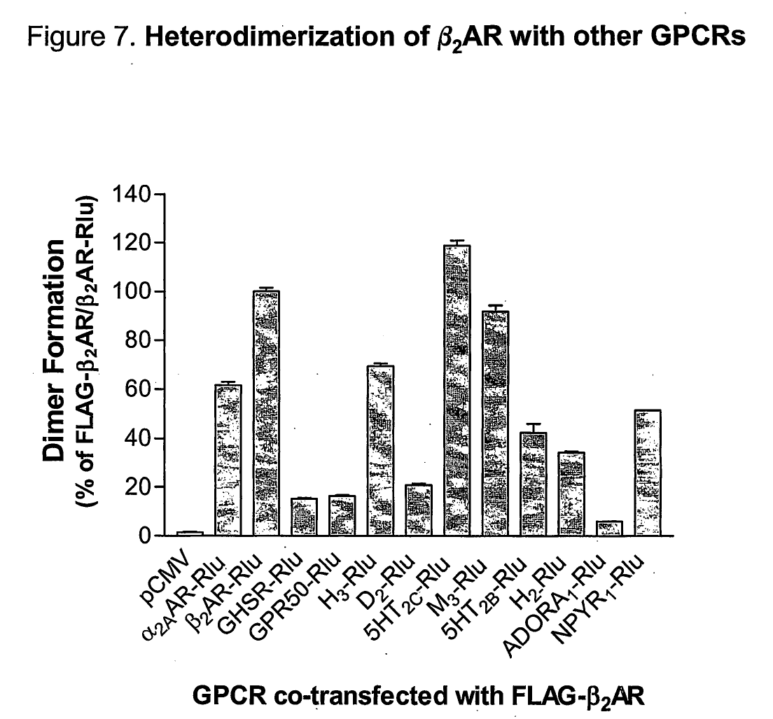

HEK293 cells were transiently transfected with FLAG-β₂AR and indicated Rlu-tagged receptor plasmids at ratio of 1:1 (w/w). Cells were harvested after about 40 h transfection and lysed as described under “Experimental methods”. 50 μl of cell lysate containing 12.5 μg total proteins and 0.25% digitonin and 50 μl of anti-FLAG antibody solution (5 μg/ml in PBS containing 1% BSA) were added into 96-well assay plate. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Heterodimeric β₂AR formation is expressed as percentage of homodimeric FLAG-β₂AR/β₂AR-Rlu which is set as 100%. Data represent one of three independent experiments, performed in triplicate. The results of these assays are shown in FIG. 7. These results show that α_{2A}AR, 5HT_{2C}, M₃, H₃, NPY₁ 5HT_{2B} and H₂ could significantly associate with -β₂AR, whereas GHSR, GPR50, D₂ and ADORA₁ had weak or no association with -β₂AR. These results are of significance because, on the basis of DNA microarray gene expression experiments, β₂AR is endogenously co-expressed with at least a) α_{2A}AR in aorta and pericardium, b) M₃ in stomach, NPYR₁ in spleen, 5HT_{2c} and H₃ in various brain sub regions, and 5HT_{2c} in certain reproductive tissues, e.g., ovary.

### Example 7

### Effect of Receptor Expression Levels on Homo- and Heterodomerization

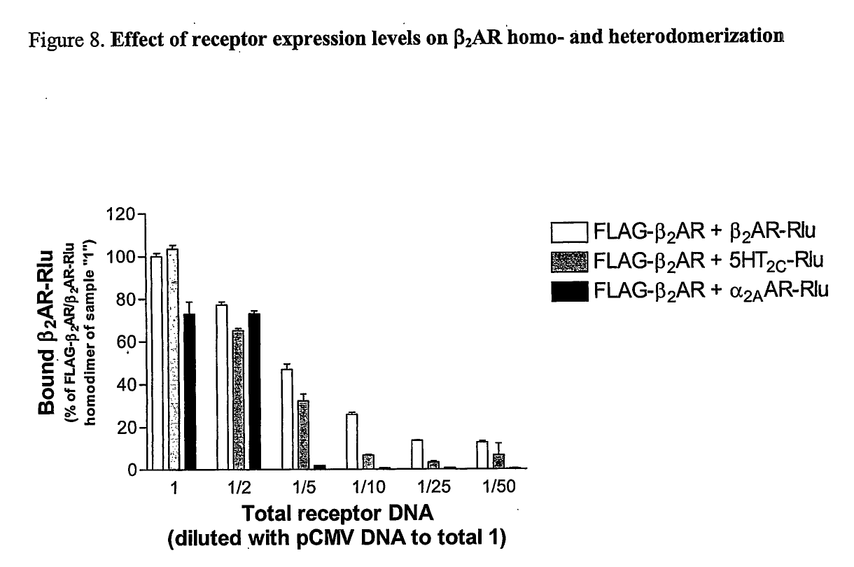

HEK₂₉₃ cells were transiently transfected with various of total receptor plasmids (The ratio of FLAG-β₂AR and indicated Rlu-tagged receptor plasmids was kept at constant 1:1 (w/w) and total plasmids (4 μg/10 cm dish) were adjusted to the same with pCMV). Cells were harvested after about 40 h transfection and lysed as described under “Experimental methods”. 50 μl of cell lysate containing 12.5 μg total proteins and 0.25% digitonin and 50 μl of anti-FLAG antibody solution (5 μg/ml in PBS containing 1% BSA) were added into assay plate. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Homo- or heterodimeric β₂AR formation is expressed as percentage of homodimeric FLAG-β₂AR/β₂AR-Rlu under normal transfection conditions (no pCMV dilution) which is set as 100%. Data represent one of three independent experiments, performed in triplicate. The results of these assays are shown in FIG. 8. These results show that receptor expression levels were proportional to transfected receptor plasmid amount as confirmed by determining receptor binding sites in a radio-ligand binding assay. Normally β₂AR expressed at 1-3 pmol per mg proteins in transfected cells under condition #1 (without pCMV dilution). Physiological expression level of β₂AR in tissues or cells are in the range of 10-200 fmol per mg proteins which corresponded to conditions between (⅕ and 1/10 dilution). These results suggest that β₂AR can form dimer under physiological expression levels.

### Example 8

### Effect of 5HT_{2C} Coexpression on Pharmacology of β₂AR

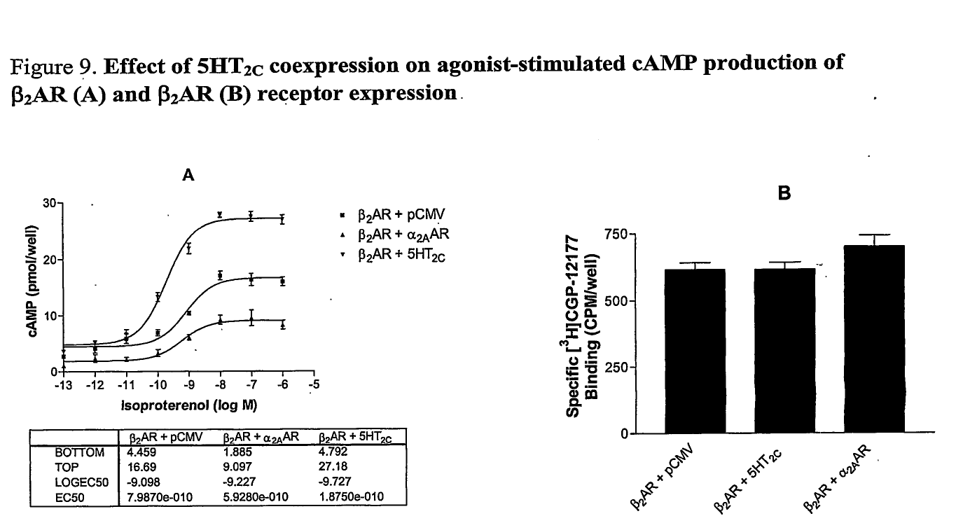

HEK293 cells were transiently transfected with wild-type β₂AR and pCMV, α₂AR (wt) or 5HT_{2C} (wt) at ratio of 1:1 (w/w). Isoproterenol-stimulated cAMP increase in intact cells expressing indicated receptors was determined by commercially available cAMP FlashPlate assay kit (NEN Life Science Products, catalog number SMP004A) under manufacturer's instructions (A). The expression levels of β₂AR was estimated by a ligand binding assay using [³H]CGP-12177 as radioligand (B). Briefly, membranes (25 μg/well) from cells transiently expressing indicated receptors were incubated in binding buffer (50 mM Tris-HCl, pH 7.4, 1 mM EDTA and 10 mM MgCl₂) with 2 nM [³H]CGP-12177, an agonist for β₂AR in the absence of (total binding) or in the presence of 100 μM isoproterenol, also an agonist of β₂AR, (non-specific binding) for 2 h prior to terminate reaction. Data represent one of three independent experiments, performed in triplicate. The results of these assays are shown if FIG. 9. “Bottom” is basal cAMP level (pmol/well); “Top” is maximal cAMP level (pmol/well); LogEC50 (logM) and EC50 (M) are indications of ligand potency. These results show that coexpression of 5HT2C potentiate agonist-stimulated cAMP production by β₂AR without significant changing receptor expression level of β₂AR.

### Example 9

### Heterodimerization of 5HT_{2C} with Other GPCRs

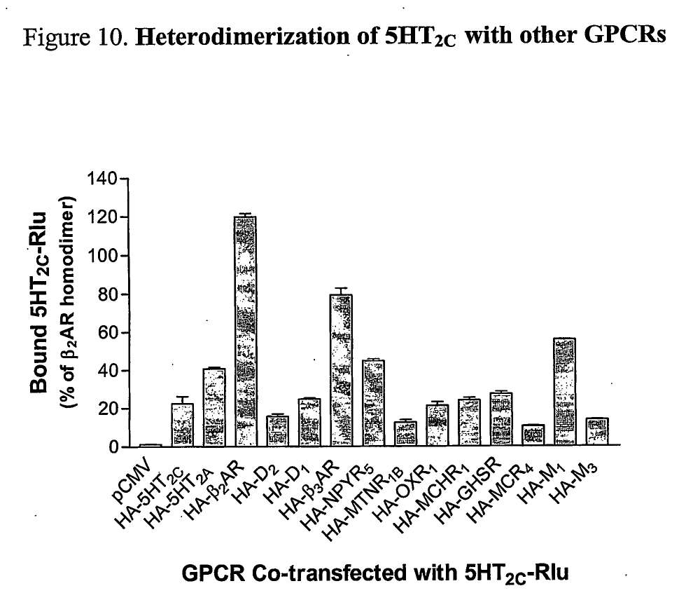

HEK293 cells were transiently transfected with 5HT_{2C}-Rlu and indicated N-terminal HA-tagged receptor plasmids at ratio of 1:1 (w/w). Cells were harvested after about 40 h transfection and lysed as described under “Experimental methods”. 50 μl of cell lysate containing 12.5 μg total proteins and 0.25% digitonin and 50 μl of anti-HA antibody solution (5 μg/ml in PBS containing 1% BSA) were added into 96-well assay plate. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. Dimeric receptor formation is expressed as percentage of homodimeric HA-β₂AR/β₂AR-Rlu which is set as 100%. Data represent one of three independent experiments, performed in triplicate. The results of these assays are shown in FIG. 10. These results show that 5HT2A, β₂AR, β₂AR M1, NPY₅ could significantly associate with 5HT_{2C}, whereas other tested receptors had weak or no association with 5HT_{2C}. These results are of significance because, on the basis of DNA microarray gene expression experiments, 5HT_{2c} is endogenously co-expressed with at least M₁, 5HT_{2A} and NPYR₅ in hypothalamus and hippocampus sub-regions of the brain.

### Example 10

### Detection of Gαs Interaction With β₂AR-Rlu

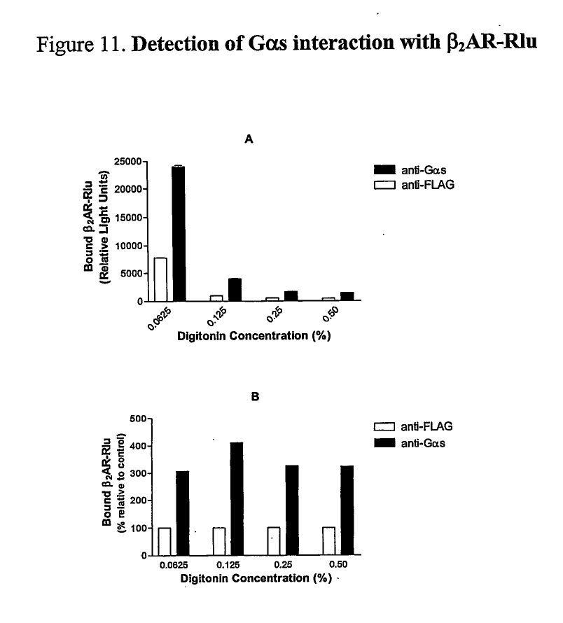

HEK293 cells were transiently transfected with β₂AR-Rlu. Cells were harvested after about 40 h transfection and lysed as described under “Experimental methods”. To protein A-coated binding plate 50 μl of cell lysate containing 12.5 μg total proteins and indicated concentrations of digitonin was added, then 50 μl of anti-FLAG or anti-Gas antibodies (5 μg/ml in PBS containing 1% BSA) was added. The assay plate was incubated under shaking at room temperature for 1-2 h. The plate was then washed six times with PBS containing 0.05% Tween-20. Finally, 100 μl of assay solution (2 μM coelenterazine in assay buffer) was added to each well and mixed prior to measuring luciferase activity using a Microbeta Counter. β₂AR-Gαs interaction was assessed by specific luciferase activity of bound β₂AR-Rlu to anti-Gαs antibody compared to non-specific binding to ant-FLAG antibody. Rough data (A) were converted to ratio of signal to noise (B). Data represent one of three independent experiments, performed in triplicate. The results of these assays are shown in FIG. 11. These results show that specific interaction of β₂AR-Rlu with Gαs could be detected by anti-Gαs antibody. Digitonin concentrations had effect on total bound-β₂AR-Rlu, but had no significant effect on ratio of signal to noise.

### Example 11

### Detection of Effect of Ligand on Receptor Dimerization

HEK293 are transiently transfected with FLAG-β₂AR and β₂AR-Rlu at a ratio of 1:1 (w/w). Cells are harvested after about 40 hr transfection and membrane is prepared as described above under “Materials and Methods”. The membrane is incubated in the binding buffer (50 mM Tris-HCl, pH 7.4, 50 mM NaCl and 10 mM MgCl₂ in the absence or in the presence of ligand (agonist or antagonist) at room temperature for 60 min. After washing, the cells on the membrane are lysed with lysate buffer containing various detergents at various concentrations for 45 min. The supernatant is collected after centrifugation. 50 μl of cell lysate containing 12.5 μg of anti-FLAG antibody solution (5 μg/ml in PBS containing 1% BSA) is added into an assay plate. The assay plate is incubated under shaking at room temperature for 1-2 hr. The plate is then washed six times with PBS containing 0.05% Tween-20. Finally, 100 μl of assay solution (2 μM coelenterazine in assay buffer) is added to each well and mixed prior to measuring luciferase activity using a Microbeta Counter.

It is evident from the above results and discussion that the subject invention provides an important new means for identifying binding partners of a GPCR, and, in particular, a system for screening chemical agent libraries to find modulators of a GPCR complex. As such, the subject methods and systems find use in a variety of different applications, including research, medical, therapeutic and other applications. Accordingly, the present invention represents a significant contribution to the art.

Applicant reserves the right to exclude any one or more GPCRs from any of the embodiments of the invention. Applicant further reserves the right to exclude any polynucleotide or polypeptide from any of the embodiments of the invention.

1. A method for detecting a GPCR-binding partner complex comprising a GPCR and a GPCR binding partner, said method comprising:
(a) culturing a cell producing a first and a second polypeptide, wherein at least one of said first and said second polypeptides is a GPCR,
(b) lysing said cell;
(c) contacting said first polypeptide with a substrate having affinity for said first polypeptide, under conditions suitable for binding of said first polypeptide to said substrate; and
(d) detecting the presence of said second polypeptide on said substrate, wherein said detecting is direct;
wherein the presence of said second polypeptide on said substrate is indicative of a GPCR-binding partner complex.

2. The method of claim 1, wherein said first polypeptide is a GPCR and said second polypeptide is a GPCR binding partner.

3. The method of claim 1, wherein said first polypeptide is a GPCR binding partner and said second polypeptide is a GPCR.

4. The method of claim 1, wherein at least one of said first and said second polypeptides is an orphan GPCR.

5. The method of claim 1, wherein at least one of said first and second polypeptides is a native GPCR.

6. (canceled)

7. The method of claim 1, wherein both of said first and said second polypeptides are GPCRs.

8-11. (canceled)

12. The method of claim 1, wherein at least one of said first and said second polypeptides is recombinant.

13-22. (canceled)

23. The method of claim 1, wherein said first and said second polypeptides are endogenously co-expressed in at least one cell type, tissue, or tissue sub-region.

24. The method of claim 1, wherein said method further comprises selecting prior to said culturing step (a) said first and said second polypeptides wherein said first and said second polypeptides are coexpressed in a least one cell type, tissue or tissue sub-region.

25. A method for detecting a GPCR-binding partner complex comprising a GPCR and a GPCR binding partner, said method comprising:
(a) culturing a cell producing a first and a second polypeptide, wherein at least one of said first and said second polypeptides is a GPCR,
(b) lysing said cell;
(c) contacting said first polypeptide with an addressable substrate having affinity for said first polypeptide, under conditions suitable for binding of said first polypeptide to said substrate in an addressable manner; and
(d) detecting the presence of said second polypeptide on said substrate, wherein said detecting is direct;
wherein the presence of said second polypeptide on said substrate is indicative of a GPCR-binding partner complex.

26. The method of claim 25, wherein said first polypeptide is a GPCR and said second polypeptide is a GPCR binding partner.

27. The method of claim 25, wherein said first polypeptide is a GPCR binding partner and said second polypeptide is a GPCR.

28. The method of claim 25, wherein at least one of said first and said second polypeptides is an orphan GPCR.

29. The method of claim 25, wherein at least one of said first and said second polypeptides is a native GPCR.

30. (canceled)

31. The method of claim 25, wherein both of said first and said second polypeptides are GPCRs.

32-35. (canceled)

36. The method of claim 25, wherein at least one of said first and said second polypeptides is recombinant.

37-51. (canceled)

52. The method of claim 25, wherein said first and said second polypeptides are endogenously co-expressed in at least one cell type, tissue, or tissue sub-region.

53. The method of claim 25, wherein said method further comprises selecting prior to said culturing step (a) said first and said second polypeptides wherein said first and said second polypeptides are coexpressed in a least one cell type, tissue or tissue sub-region.

54. A method of identifying whether a candidate polypeptide is a binding partner for a GPCR, comprising the step of detecting a GPCR-binding partner complex comprising said candidate polypeptide and said GPCR according to the method of claim 1 or claim 25, wherein detection of said complex is indicative of said candidate polypeptide being a binding partner of said GPCR.

55. A method for detecting a GPCR-binding partner complex, said method comprising:
(a) culturing a plurality of cells, each cell producing a first and a second polypeptide, wherein at least one of said first and said second polypeptides is a GPCR, and wherein each cell produces a different GPCR;
(b) lysing said cells;
(c) contacting said first polypeptide from each cell with an addressable substrate having affinity for said first polypeptide, under conditions suitable for binding of said first polypeptide to said substrate at an address specific for said cell; and
(d) detecting the presence of said second polypeptide on said substrate, wherein said detecting is direct;
wherein the presence of said second polypeptide at an address on said substrate is indicative of a GPCR-binding partner complex comprising said first polypeptide and said second polypeptides produced by the cell having said address.

56. The method of claim 55 wherein said plurality of cells is at least 2, at least 5, at least 10, at least 15, at least 20, at least 25, at least 50, or at least 100 cells.

57. The method of claim 55, wherein said first and said second polypeptides are endogenously co-expressed in at least one cell type, tissue, or tissue sub-region.

58. The method of claim 55, wherein said method further comprises selecting prior to said culturing step (a) said first and said second polypeptides wherein said first and said second polypeptides are coexpressed in a least one cell type, tissue or tissue sub-region.

59. A method for detecting a GPCR-binding partner complex, said method comprising:
(a) culturing a cell, said cell producing a first and a plurality of a second polypeptide, wherein each of said plurality of said second polypeptide is different and wherein at least one of said first and said plurality of said second polypeptide is a GPCR;
(b) lysing said cell;
(c) contacting said first polypeptide with a substrate having affinity for said first polypeptide, under conditions suitable for binding of said first polypeptide to said substrate; and
(d) detecting the presence of said second polypeptide on said substrate, wherein said detecting is direct;
wherein the presence of said second polypeptide on said substrate is indicative of at least one GPCR-binding partner complex comprising said first polypeptide and said second polypeptide.

60. The method of claim 59 wherein said GPCR-binding partner complex is detected, further comprising repeating steps (a) to (d) one or more times with subsets of said plurality of said second polypeptide, said subsets encompassing said plurality, until a GPCR-binding partner complex is detected from at least one cell producing a said first polypeptide and a single said second polypeptide.

61. The method of claim 59 wherein said plurality of said second polypeptides is at least 2, at least 5, at least 10, at least 15, at least 20, at least 25 said, at least 50, or at least 100 of second polypeptide.

62. (canceled)

63. The method of claim 59, wherein said first and second polypeptides are endogenously co-expressed in at least one cell type, tissue, or tissue sub-region.

64. The method of claim 59, wherein said method further comprises selecting prior to said culturing step (a) said first and said second polypeptides wherein said first and said second polypeptides are coexpressed in a least one cell type, tissue or tissue sub-region.

65. The method of claim 55 or claim 59, wherein said first polypeptide is a GPCR and said second polypeptide is a GPCR binding partner.

66. The method of claim 55 or claim 59, wherein said first polypeptide is a GPCR binding partner and said second polypeptide is a GPCR.

67. The method of claim 55 or claim 59, wherein at least one of said first and said second polypeptides is an orphan GPCR.

68. The method of claim 55 or claim 59, wherein at least one of said first and said second polypeptide is a native GPCR.

69. (canceled)

70. The method of claim 55 or claim 59, wherein both of said first and said second polypeptides are GPCRs.

71-74. (canceled)

75. The method of claim 55 or claim 59, wherein at least one of said first and second polypeptides is recombinant.

76-85. (canceled)

86. A method for identifying whether a candidate agent modulates binding of a GPCR to a binding partner for the GPCR, said method comprising the step of determining whether there is a difference in the amount of a GPCR-binding partner complex comprising said GPCR and said binding partner detected according to the method of claim 1 or claim 25, wherein said difference is determined for contacting or not contacting said candidate agent with said first and said second polypeptides prior to said detecting step (d) of said method and wherein an alteration in said amount of said second polypeptide detected on said affinity substrate is indicative of an agent that modulates binding of said GPCR to said binding partner for the GPCR.

87. The method of claim 86, wherein said first and said second polypeptides are contacted with said candidate agent during step (a), step (b) or step (c).

88. The method of claim 86, wherein said candidate agent is a small molecule, a peptide, a ligand for said GPCR, or an antibody.

89. The method of claim 86, wherein said modulator reduces said binding of a GPCR to a binding partner for the GPCR by more than about 10%, more than about 20%, more than about 30%, more than about 40%, or more than about 50% as compared to said binding in the absence of said agent.

90. The method of claim 86, wherein said modulator increases said binding of a GPCR to a binding partner for the GPCR by more than about 10%, more than about 25%, more than about 50%, more than about 100%, more than about 200%, more than about 300%, more than about 400%, or more than about 500.

91. The method according to any one of claims 1, 25, 55 and 59 wherein said first and said second polypeptides are both mammalian.

92. The method according to any one of claims 1, 25, 55 and 59 wherein said first and second polypeptides are both human.

93. A composition comprising an addressable affinity substrate, said addressable affinity substrate comprising a plurality of addresses having affinity for different GPCRs.

94-97. (canceled)

98. A composition comprising a cell, said cell producing a first and a second polypeptide, wherein said first and said second polypeptides are a GPCR and a GPCR binding partner, and wherein said first polypeptide comprises an affinity tag and said second polypeptide is fused to a reporter protein.

99-115. (canceled)

116. A composition comprising a library of GPCRs, wherein said library comprises a plurality of pairs of isolated polynucleotide wherein both a first and a second said polynucleotide of said pair comprises a first nucleotide sequence encoding the same GPCR, said encoded GPCR of one said pair being different from said encoded GPCR of any other said pair, and wherein said first polynucleotide of said pair further comprises a second nucleotide sequence encoding an in-frame affinity tag and said second polynucleotide of said pair further comprises a third nucleotide sequence encoding an in-frame reporter protein.

117. The composition of claim 116 wherein said affinity tag is an epitope tag and said reporter protein is luciferase.

118. The composition of claim 116 wherein said library comprises at least about 50 said pairs, at least about 100 said pairs, at least about 200 said pairs, at least about 300 said pairs, at least about 400 said pairs, or at least about 500 said pairs.

119. The composition of claim 116 wherein said encoded GPCR is native.

120. The composition of claim 116 wherein said encoded GPCR is mammalian.

121. The composition of claim 116 wherein said encoded GPCR is human.
